(19) United States
(12) Patent Application Publication
(10) Pub. No.: US 2024/0252644 A1
(43) Pub. Date: Aug. 1, 2024

(54) PERSON-TAILORED T CELL COMPOSITION TARGETING MERKEL CELL CARCINOMA

(71) Applicants: Danmarks Tekniske Universitet, Kongens Lyngby (DK); University of Washington, Seattle, WA (US)

(72) Inventors: Ulla Kring Hansen, Kongens Lyngby (DK); Sine Reker Hadrup, Kongens Lyngby (DK); Paul Tuan-Xuan Nghiem, Kongens Lyngby (DK)

(21) Appl. No.: 18/565,040

(22) PCT Filed: Jun. 2, 2022
(86) PCT No.: PCT/EP2022/065049

§ 371 Date: Nov. 28, 2023

Related U.S. Application Data
(60) Provisional application No. 63/195,938, filed on Jun. 2, 2021.

(30) Foreign Application Priority Data
Jun. 22, 2021 (EP) 21180985.0

(51) Int. Cl. A61K 39/00; C07K 14/005; C07K 14/74; C12N 5/0783

(52) U.S. Cl. CPC A61K 39/464838; A61K 39/4611; C07K 14/005; C07K 14/70539; C12N 5/0636; C12N 2501/2302; C12N 2501/2321; C12N 2502/1157; C12N 2710/22022

(57) ABSTRACT

The present invention relates to a method for producing a person-tailored T cell composition by in vitro stimulation and expansion of T cells comprising the steps of i) providing at least one identified HLA haplotype from a subject; ii) preparing at least one APC comprising at least one HLA haplotype corresponding to said at least one identified HLA haplotype; and at least one antigenic peptide matched to said at least one HLA haplotype; wherein said at least one antigenic peptide comprises an epitope from Merkel cell polyomavirus, said epitope originates from large T antigen (LTA), small T antigen (STA) or the shared region (CT) of LTA and STA; iii) providing a sample comprising T cells, iv) contacting said sample with an expansion solution comprising at least one APC as prepared in step ii, v) stimulating and expanding T cells with specificity for said at least one antigenic peptide comprised on at least one APC in culture, and optionally harvesting the T cells from the culture, to obtain a person-tailored T cell composition.

### STATEMENT OF US GOVERNMENT SUPPORT

This invention was made with government support under Grant No. P01 CA225517, awarded by the National Institutes of Health. The government has certain rights in the invention.

### TECHNICAL FIELD OF THE INVENTION

The present invention relates to a method for producing a person-tailored T cell composition directed towards subjects in need of treatment targeting Merkel Cell Carcinoma. In particular, the invention relates to a method using APC for stimulating and expanding T cells targeting Merkel Cell Carcinoma.

### BACKGROUND OF THE INVENTION

Merkel Cell carcinoma (MCC) is a rare but highly aggressive human skin cancer with poor prognosis for nodal and distant advanced disease. The oncovirus Merkel cell polyomavirus (MCPyV) is associated with at least 80% of MCC cases. Although MCPyV is commonly found in humans, its oncogenic potential requires integration into the host genome and truncation of the Large T antigen (LTA), which leads to inactivation of viral replication and persistent expression of the viral T antigens (T-Ag; LTA and small T antigen (STA)), which drives oncogenesis.

The adaptive immune system plays a key role in MCC tumor control and increased survival has been associated with intratumoral levels of both CD3+ and CD8+ lymphocytes. Checkpoint inhibitors blocking the programmed death 1 (PD-1) pathway have proven highly effective, and are now standard first-line therapy for advanced MCC. Nevertheless, for half of the patients, this treatment strategy is insufficient. Instead, T-cell-based therapies, such as cell therapies, could be attractive strategies, but require identification of CD8+ T-cell epitopes within T-Ag that serve as targets for tumor cell elimination.

CD8+ T-cell epitopes are matched to specific HLA haplotypes for correct stimulation and activation of T-cells. The specific pattern of HLA haplotypes varies from individual to individual. Accordingly relevant T-cell epitopes will vary from individual to individual why T-cell based therapy needs to be person-tailored in order to be effective.

Hence, an improved method and kit for quickly and efficiently obtaining T-cell compositions directed towards a specific subject in need of treatment would be advantageous, and in particular a more efficient and/or reliable composition for use in treatment of MCC and MCC tumor control would be advantageous.

### SUMMARY OF THE INVENTION

Thus, an object of the present invention relates to the provision of a method and a kit in parts for easy generation of a person-tailored T cell composition.

In particular, it is an object of the present invention to provide a composition that solves the above mentioned problems of the prior art with insufficient effect on tumor control observed by using known checkpoint inhibitors but which may be obtained and used for targeting MCC in all subjects in need of treatment.

Thus, one aspect of the invention relates to a method for producing a person-tailored T cell composition by in vitro stimulation and expansion of T cells comprising the steps of
i. providing at least one identified HLA haplotype from a subject;
ii. preparing at least one APC comprising at least one HLA haplotype corresponding to said at least one identified HLA haplotype; and at least one antigenic peptide matched to said at least one HLA haplotype; wherein said at least one antigenic peptide comprises an epitope from Merkel cell polyomavirus, said epitope originates from large T antigen (LTA), small T antigen (STA) or the shared region (CT) of LTA and STA;
iii. providing a sample comprising T cells,
iv. contacting said sample with an expansion solution comprising at least one APC as prepared in step ii,
v. stimulating and expanding T cells with specificity for said at least one antigenic peptide comprised on at least one APC in culture, and optionally harvesting the T cells from the culture, to obtain a person-tailored T cell composition.

Another aspect of the present invention relates to a person-tailored T cell composition obtained by a method as described herein.

A further aspect of the present invention relates to a person-tailored T cell composition as described herein for use as a medicament.

A still further aspect of the present invention relates to a person-tailored T cell composition as described herein for use in the prevention, alleviation and/or treatment of Merkel Cell Carcinoma.

An even further aspect of the present invention relates to a kit for expansion of T cells, the kit in parts comprising:
i) at least one APC having a given HLA haplotype;
ii) at least one antigenic peptide wherein said antigenic peptide comprises an epitope from Merkel cell polyomavirus, said epitope originates from large T antigen (LTA), small T antigen (STA) or the shared region (CT) of LTA and STA; and
   wherein said at least one APC and said at least one antigenic peptide are configured to be combined by combining said given HLA haplotype with a matched antigenic peptide.

### BRIEF DESCRIPTION OF THE FIGURES

FIG. 1 shows (A) a schematic overview of the DNA barcode-labelled multimer technology used in the screening of MCC patient samples for T cell recognition of LTA or STA-derived epitopes. First potential CD8+ T cell epitopes were selected based on an in silico prediction of 9- and 10-mer peptides with binding capacities to 33 different HLA haplotypes (LTA and STA sequence & Selected HLA haplotypes). MHC binders were defined by an eluted ligand rank score<2 using NetMHCpan 4.0 (Prediction of MHC class I binding & Peptide pool). DNA barcode-labelled multimers were generated, where a unique DNA barcode defines the peptide specificity and used for staining MCC patient samples (DNA barcoded pMHC multimer. Each pMHC is assigned a unique DNA barcode). Multimer panels were generated with >100 peptide specificities simultaneously. Multimer-binding CD8+ T cells were selected (Staining of patients's samples with multimer pool) and sorted (sorting of multimer CD8+ cells) based on their positive PE signal. Their epitope specificity was revealed through amplification (amplification of DNA barcodes) and sequencing of the associated DNA barcodes (sequencing and data analysis). DNA barcodes enriched in the sorted T cell fraction compared to baseline level with an FDR<0.1% were defined as being recognized in the given sample (enriched DNA barcodes in the sorted fraction define the T cell specificity). (B-D) A collection of all screening data in MCC patients resulted in a total of 76 recognized T cell epitopes presented in the context of 20 different MHC haplotypes. Epitope-specific T cells were detected in 7.1-100% of the screened MCC patients divided as either HLA-A (B), HLA-B (C), or HLA-C (D) restricted epitopes.

FIG. 2 shows the detected LTA or STA epitopes recognized by T cells in the DNA barcode-labelled multimer screen mapped along the sequences of CT, truncated LTA, and STA. The epitope sequences are colored based on HLA restriction and divided according to HLA class as HLA-A (A), -B (B), and -C (C) restricted epitopes.

FIG. 3 shows a comparison between the number of antigen-specific T cell populations detected in the MCC patient cohort (MCC, circles) and a healthy donor cohort (HD, triangles) for either recognition of LTA or STA-derived epitopes (black) or VP1-derived epitopes (grey).

FIG. 4 shows the expansion of LTA/STA-specific T cells from MCC patients' PBMC samples using a magnetic selection of antigen-specific cells. The schematic overview (A) shows how T cells 1 are labelled with HLA-matched, PE-labelled pMHC tetramers 3 coupled to anti-PE magnetic beads 5, placed in a magnetic field for selection of tetramer binding cells (magnetic separation), eluted (elution of labeled cells) and cultured for two weeks (2 weeks expansion). The pMHC specificity was revealed by combinatorially encoded fluorescently labeled pMHC tetramers given a dual-color signal detectable by flow cytometry, and presented as double positive events in dot plots (X and Y axis) (B).

FIG. 5 shows the expansion of LTA/STA-specific T cells (CD8+) from a single MCC patient's PBMC sample using artificial antigen presenting cell (aAPC) scaffold 7. The top dot plot (part ½) shows the DNA barcode-labelled multimer screen where multimer positive cells (X axis) were sorted to reveal the LTA/STA specificity given by the associated DNA barcode. The patients' PBMCs were then expanded with aAPC scaffolds, which is comprised of a dextran backbone coupled with the pMHC complex of interest and cytokines for co-simulation of the T cells in the ratio 1:24:6:6. The PBMCs were cultured for two weeks (2 weeks expansion) with aAPC scaffolds feeded at day 0, 3, 6, and 9. Following harvest on day 14 the cells were stained with APC labelled multimers (a single-color tetramer) to reveal the LTA/STA-specific T cells (X axis) by flow cytometry (bottom dot plot (part 2/2)).

FIG. 6 shows the functional capacity of LTA/STA-specific T cells in response to MCC tumor cell recognition. PBMC samples from two MCC patients were expanded using the magnetic selection of antigen-specific cells, followed by stimulation with HLA-matched Merkel Cell Polyomavirus (MCPyV)-positive or -negative tumor cell lines (TCLs). The cells were stained with DNA barcode-labeled pMHC multimers and antibodies against intracellular cytokines and sorted according to their cytokine secretion profile (IFNγ and/or TNFα secretion (ICS^{pos}) or no cytokine secretion (ICS^{neg})). The pMHC specificity was revealed by sequencing of the pMHC multimer associated DNA barcode (A). The epitope specificities of the functionally activated cells were revealed by sequencing of the co-attached DNA barcodes present in both sorted pools (B-C). The bar plots display −Log₁₀(p) of significantly enriched DNA barcodes, hence pMHC specificities recognized by T cells. The dotted lines represent the selected threshold of FDR=0.1% (p=0.001). Black bars represent epitopes recognized by T cells in the ICS^{pos} fraction and grey bars represent epitopes recognized by T cells in the ICS^{neg} fraction.

FIG. 7 shows the expansion of LTA/STA-specific CD8+ T cells from 5 MCC patients' PBMC samples using aAPC scaffold 7. The PBMCs were expanded with person-tailored aAPC scaffolds based on the patients' HLA haplotypes, which are comprised of a dextran backbone coupled with the pMHC complex of interest and cytokines for co-simulation of the T cells in the ratio 1:24:6:6. The PBMCs were cultured for two weeks (2 weeks expansion) with aAPC scaffolds added on day 0, 3, 6, and 9. Following harvest on day 14 the expanded and un-expanded cells were stained with combinatorial encoded fluorescently labeled pMHC tetramers to reveal the LTA/STA-specific T cells as dual-color positive by flow cytometry. Based on these measurements the number of LTA/STA-specific CD8+ T cells was calculated for all patients (summarized graph, A). Examples of two single epitope expansions (z1440 and z1253, respectively) are shown as double-positive events in dot plots (B).

FIG. 8 compares the phenotype of the LTA/STA-specific CD8+ T cells from 5 patients expanded with person-tailored aAPC scaffolds (D14) against the un-expanded LTA/STA-specific cells (D0), from the same patients. The bar plots present either (A) cell type (effector memory (EM), central memory (CM), terminally differentiated effector memory (TEMRA), or naïve T cells), or (B) level expression of different activation, proliferation, and exhaustion markers.

FIG. 9 compares the capacity for tumor cell killing (functional activity) of aAPC scaffold expanded (D14) and un-expanded (D0) PBMCs from a single patient . The co-culture between target, non-target, and effector cells was set up at different effector:true target (E:T) ratios (X axis), and the percent specific lysis of the true target TCL (WAGA) was measured after 48 hours (Y axis).

The present invention will now be described in more detail in the following.

### DETAILED DESCRIPTION OF THE INVENTION

### Definitions

Prior to discussing the present invention in further details, the following terms and conventions will first be defined:

### Person-Tailored T Cell Composition

In the present context, the term “person-tailored T cell composition” refers to a T cell composition, which is tailored to an epitope-selection based on the individual HLA profile of that specific subject as described by the present invention. In one embodiment, the T cell composition is based on the patient's own T cells.

### Subject

In the present context, the term “subject” refers to a human.

### HLA

In the present context, the term “HLA” refers to human leukocyte antigen (HLA) gene complex that is encoded by the major histocompatibility complex (MHC) in humans. MHC and HLA are used interchangeably herein.

The main function of this protein complex is to bind antigenic peptides derived from pathogens and display them on the cell surface for recognition by the appropriate T-cells.

HLA molecules consist of an alpha-chain (heavy chain) produced by MHC genes and a beta-chain (light chain or β2-microglubulin) produced by the β2-microglubulin gene.

The heavy chain consists of three domains denoted alpha-1, alpha-2 and alpha-3, respectively. The alpha-1 domain is located next to the non-covalently associated β2-microglubulin. The alpha-3 domain is a transmembrane domain, which anchors the HLA molecule in the cell membrane. Together, the alpha-1 and alpha-2 domains form a heterodimer containing a peptide-binding groove which binds a specific antigenic peptide. The amino acid sequence of the peptide-binding groove is the determinant as to which specific antigenic peptide is bound to the HLA molecule.

There exist three major types of HLA (classified in MHC class I) that are coded in the gene loci for HLA-A, HLA-B, and HLA-C. For each of these three groups there are many different specific HLA proteins as HLA genes are highly polymorphic i.e. having several different alleles.

### HLA Haplotype

In the present context, the term “HLA haptotype” refers to the specific HLA protein encoded for. An examples of an HLA haplotype is A*0101. Another example of an HLA haplotype is B*0801. A further example of an HLA haplotype is C*0501. The sequences of the HLA haplotypes are publicly available from the GenBank.

### HLA Type

In the present context, the term “HLA type” refers both to the three major types of HLA i.e. HLA-A, HLA-B and HLA-C as well as the HLA haplotype. In one embodiment, the HLA type is the HLA haplotype.

### Barcode

In the present context, the term “barcode” refers to a short artificial section of DNA attached to individual compounds in a mixture of compounds used for unique identification by subsequent PCR amplification.

### Antigenic Peptide

In the present context, the term “antigenic peptide” refers to a peptide that is capable of binding to a human leukocyte antigen (HLA) gene complex molecule to form a peptide-HLA (pHLA) complex. The pHLA complex can present the antigenic peptide to immune cells to induce a T-cell receptor dependent immune response.

The antigenic peptide is matched with a specific HLA haplotype to obtain correct binding and display of the antigenic peptide in the groove of the HLA molecule.

In one embodiment, the antigenic peptide comprises an epitope originating from large T antigen, small T antigen or the shared region of large T antigen and small T antigen. In a further embodiment, the antigenic peptide consists of an epitope originating from large T antigen, small T antigen or the shared region of large T antigen and small T antigen.

### Epitope

In the present context, the term “epitope” means the antigenic determinant recognized by the TCR of the T cell. The epitope presented by the pHLA is highly specific for any foreign substance and the interaction with the TCR ensures effective expansion and functional stimulation of the specific T cells in a peptide-HLA-directed fashion.

### APC

In the present context, the term “APC” refers to natural occurring antigen-presenting cells as well as artificial APCs.

Examples of natural occurring APCs are autologous APCs i.e. obtained from the same subject as the one to be treated, and allogenic APCs i.e. genetically different but belonging to the same species as the subject to which it is to be used as well as having identical HLA haplotypes.

### Artificial APC

In the present context, the term “artificial APC” or “aAPC” refers to non-natural APCs that mimic the natural interaction between the TCR and the specific antigenic peptide presented by the major histocompatibility complex (MHC). Thus, it means an assembly of molecules essential for functioning similar to an antigen presenting cell.

Examples of aAPC are aAPC scaffolds, aAPC beads and cellular aAPC.

### AAPC Scaffold

In the present context, the term “aAPC scaffold” refers to an aAPC comprising a polymeric backbone onto which HLA molecules and T cell affecting molecules, such as cytokines and co-stimulatory molecules are fixed. In one embodiment, the aAPC scaffold is soluble.

The polymeric backbone may be of a material selected from polysaccharides, vinyl polymers, poly ethylene glycol, poly propylene glycol, strep-tactin, poly-streptavidin, biotin-binding proteins and polyhistidine-binding polymers.

### T Cell Affecting Molecule

In the present context, the term “T cell affecting molecule” refers to any molecule that has a biological effect on a T cell. Biological effects include, but are not limited to, proliferation, differentiation and stimulation of T cells.

Thus, T cell affecting molecules may be utilized for expanding and functionally manipulating a T cell population to obtain the desired differentiation resulting in high specificity, high killing capacity, high in vivo expansion and survival properties. T cell affecting molecules include, but are not limited to, cytokines, co-stimulatory molecules and adhesion molecules.

### Cytokine

In the present context, the term “cytokine” means an immune-regulatory molecule that affects expansion, survival and effector function of stimulated T cells. Cytokines include chemokines, interferons, interleukins, lymphokines, and tumor necrosis factors.

### Gamma-Chain Receptor Cytokines

In the present context, the term “gamma-chain receptor cytokines” refers to the group of cytokines that bind to a corresponding cytokine receptor comprising the common gamma-chain subunit. The common gamma-chain (γ_{c}) receptor is also known as CD132 or interleukin-2 receptor subunit gamma (IL-2RG). One common denominator for the gamma-chain receptor cytokines is that they all deliver their intracellular signal through the shared gamma-chain receptor and influence T-cell activation and differentiation.

### Co-Stimulatory Molecule

In the present context, the term “co-stimulatory molecule” means a molecule that upon interaction with T cells enhances T cell response, proliferation, production and/or secretion of cytokines, stimulates differentiation and effector functions of T cells or promotes survival of T cells relative to T cells not contacted with a co-stimulatory molecule. Examples of co-stimulatory molecules include, but are not limited to, B7.1, B7.2, ICOS, PD-L1, a-galactosylceramide, CD3, CD4, CD5, CD8, CD9, CD27, CD28, CD30, CD69, CD134 (OX40), CD137 (4-1BB), CD147, CDw150 (SLAM), CD152 (CTLA-4), CD153 (CD30L), CD40L (CD154), Fas (CD95), CD40, CD48, CD70, and CD72.

### Adhesion Molecule

In the present context, the term “adhesion molecule” refers to molecules that induce adhesion between the APCs and T cells. Adhesion molecules include, but are not limited to, ICAM-1, ICAM-2, GlyCAM-1, CD34, anti-LFA-1, anti-LFA-2 (CD2), LFA-3 (CD58), anti-CD44, anti-beta-7, CXCR4, CCR5, anti-selectin L, anti-selectin E, and anti-selectin P.

### Merkel Cell Carcinoma

In the present context, the terms “Merkel cell carcinoma” or “MCC”, which are used interchangeably, refer to a rare, aggressive form of skin cancer with a high risk of returning and spreading.

### Merkel Cell Polyomavirus

In the present context, the terms “Merkel cell polyomavirus”, “MCV” or “MCPyV”, which are used interchangeably, refer to a human viral pathogen suspected to cause the majority of cases of Merkel cell carcinoma as around 80% of MCC tumors have been found to be infected with MCV. It is also known as Human polyomavirus 5.

MCV is a small non-enveloped DNA virus encoding characteristic polyomavirus genes including large T antigen, small T antigen and viral capsid proteins. The T antigens are known oncoproteins. Multiple different proteins are formed depending on the splicing pattern of the gene.

### LTA

In the present context, the term “LTA” refers to large T antigen also known as large tumor antigen and is a known oncoprotein. It is expressed early in the infectious cycle and is essential for viral proliferation. Its two primary functions relate to replication of the viral genome and dysregulation of the cell cycle of the host.

### STA

In the present context, the term “STA” refers to small T antigen also known as small tumor antigen and is a known oncoprotein. It is expressed early in the infectious cycle and is usually not essential for viral proliferation. It is known to interact with host cell proteins and is important for replication.

### CT

In the present context, the term “CT” refers to the overlapping gene region between LTA and STA. This overlapping region is formed as STA is expressed from a gene that overlaps with LTA whereby the two proteins share an N-terminal Dna-like domain but have distinct C-terminal regions.

### Expansion Solution

In the present context, the term “expansion solution” refers to a solution comprising an APC for use in expansion of T cells with specificity for the APC. The expansion solution may further comprise other entities that support expansion, differentiation and stimulation of the T cells, e.g. the expansion solution may comprise additional cytokines, co-stimulatory molecules or adhesion molecules in addition to those potentially immobilized on an aAPC.

### Biological Sample

In the present context, the term “biological sample” refers to a sample obtained from the subject to be treated, which sample can be used for the HLA profiling of that subject. The biological sample is not limited to any specific source but may be extracted from blood, tumor and lymphoid tissues.

### Sample Comprising T Cells

In the present context, the term “sample comprising T cells” refers to a solution extracted from a subject, with the solution comprising a population of T cells. The sample is not limited to any specific source, but may be extracted e.g. from blood, a tissue or a body fluid. The T cell population may contain T cells with different specificities.

### Clinically Relevant Number

In the present context, the term “clinical relevant number” refers to the number of cells necessary for fighting a disease. The absolute value of the clinical relevant number of cells varies depending on the disease as well as the stage of the disease and characteristics of tumor lessions. The number of cells available before re-introduction into a patient may be in the range of 10⁴-10¹² cells per administration, such as 10⁵-10¹⁰ cells per administration, such as 10⁶-10⁹ cells per administration.

### Pharmaceutical Composition

In the present context, the term “pharmaceutical composition” refers to a composition comprising an expanded T cell population obtained according to the invention, suspended in a suitable amount of a pharmaceutical acceptable diluent or excipient and/or a pharmaceutically acceptable carrier.

### Pharmaceutically Acceptable

In the present context, the term “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

### Adjuvant

In the present context, the term “adjuvant” refers to a compound or mixture that enhances the immune response to an antigen. An adjuvant can serve as a tissue depot that slowly releases the antigen and as a lymphoid system activator, which non-specifically enhances the immune response. Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum. Preferably, the adjuvant is pharmaceutically acceptable.

### Excipient

In the present context, the term “excipient” refers to a diluent, adjuvant, carrier, or vehicle with which the composition of the invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

### Method for Producing a Person-Tailored T Cell Composition

T cells play a crucial role in the immune response, where they recognize and respond to foreign substances by interacting with antigen presenting cells (APC), displaying antigenic peptides of the foreign substance in complex with HLA molecules (pHLA). The T cells are very specific and express only a single specificity of T cell receptor (TCR), thereby allowing the T cell only to recognize and respond to a single specific pHLA molecule. When the T cells are first primed to develop receptors of a specific combination of antigen and HLA molecule, they will not subsequently be able to recognize other specificities. This specialization of the T cell is called MHC restriction and can be utilized to preferentially expand T cells of chosen specificity without direct stimulation of irrelevant T cells to obtain an expanded T cell population, primarily comprising expanded T cells with the chosen specificity.

The interplay between specific antigenic peptides and specific HLAs makes it crucial that the relevant HLAs are paired with the relevant antigenic peptides to expand an efficient T cell population. Taken together with the huge variability of the HLAs this leads to a complex puzzle to be solved in order to obtain a T cell composition effective against e.g. oncoproteins in a specific subject. Several factors thus need to be fulfilled to be able to efficiently treat these subjects.

In a first aspect, the present invention relates to a method for producing a person-tailored T cell composition by in vitro stimulation and expansion of T cells comprising the steps of
i. providing at least one identified HLA haplotype from a subject;
ii. preparing at least one APC comprising at least one HLA haplotype corresponding to said at least one identified HLA haplotype; and at least one antigenic peptide matched to said at least one HLA haplotype; wherein said at least one antigenic peptide comprises an epitope from Merkel cell polyomavirus, said epitope originates from large T antigen (LTA), small T antigen (STA) or the shared region (CT) of LTA and STA;
iii. providing a sample comprising T cells,
iv. contacting said sample with an expansion solution comprising at least one APC as prepared in step ii,
v. stimulating and expanding T cells with specificity for said at least one antigenic peptide comprised on at least one APC in culture, and optionally harvesting the T cells from the culture, to obtain a person-tailored T cell composition.

Hereby, expanded T cells can easily be obtained that are matched to the particular subject. The HLA haplotype is easily identified from the particular subject and may in some cases already be known. Based on the HLA haplotype specific for a particular subject, an APC can be created, which is able to stimulate and expand T cells directed against cells expressing antigenic peptides from the Merkel Cell polyomavirus and following treat the Merkel Cell Carcinoma of this particular subject. This is obtained by stimulating and expanding T cells specific for the APC prepared in step ii). Hereby, a person-tailored T cell composition may be obtained. The stimulated and expanded T cells from step v may optionally be harvested.

The APC to be created is based on the combination of HLA haplotype(s) with matched antigenic peptides, hereby forming a pHLA complex. Thus, it is to be understood that the antigenic peptides are bound to the HLA molecules forming pHLA complexes.

The HLA haplotype(s) on the at least one APC corresponds to at least one HLA haplotypes of the subject. Thus, as an example if the following HLA haplotypes are identified for the subject: A*0101, B*4402 and C*0401, e.g. A*0101 may be comprised on the at least one APC.

The present invention identifies several antigenic peptides matched to specific HLA haplotypes. Accordingly, by knowing the HLA haplotype of the subject, an APC can be created having corresponding HLA haplotypes and antigenic peptides known to match to this particular HLA haplotype without the necessity of identifying epitopes from the individual subjects. Epitopes that subsequently need to be optimised. Thus, it has surprisingly been found that an efficient person-tailored T cell composition can be obtained by a simple method providing the HLA haplotype of the subject.

### Antigenic Peptides

The epitopes of the antigenic peptides all originate from LTA, STA and CT. A total of 76 epitopes have been identified and matched to different HLA haplotypes (Table 1). The antigenic peptides may be identical to the epitopes as listed in Table 1 or they may comprise further amino acids in addition to the amino acid sequences of the epitopes. Thus, in one embodiment said at least one antigenic peptide comprises or consists of an epitope from Merkel cell polyomavirus. In a further embodiment, said at least one antigenic peptide comprises or consists of an epitope selected from the group consisting of SEQ ID NO: 1-76.

When the antigenic peptide comprises an epitope selected from the group consisting of SEQ ID NO: 1-76, it may comprise further amino acids. In one embodiment, the antigenic peptide has a length of at the most 20 amino acids, such as 18 amino acids, like 16 amino acids, such as 14 amino acids, like 12 amino acids, such as 10 amino acids.

In a further embodiment,
i. said at least one antigenic peptide comprises or consists of an epitope being selected from the group consisting of SEQ ID NOS: 1-2, if said HLA haplotype is A*0101;
ii. said at least one antigenic peptide comprises or consists of an epitope being SEQ ID NO: 3, if said HLA haplotype is A*0201;
iii. said at least one antigenic peptide comprises or consists of an epitope being selected from the group consisting of SEQ ID NOs: 4-7, if said HLA haplotype is A*0301;
iv. said at least one antigenic peptide comprises or consists of an epitope being selected from the group consisting of SEQ ID NOS: 8-9, if said HLA haptotype is A*1101;
v. said at least one antigenic peptide comprises or consists of an epitope being selected from the group consisting of SEQ ID NOs: 10-21, if said HLA haplotype is A*2402;
vi. said at least one antigenic peptide comprises or consists of an epitope being selected from the group consisting of SEQ ID NOS: 22-23, if said HLA haplotype is A*2601;
vii. said at least one antigenic peptide comprises or consists of an epitope being SEQ ID NO: 24, if said HLA haplotype is A*3001; and/or
viii. said at least one antigenic peptide comprises or consists of an epitope being selected from the group consisting of SEQ ID NOs: 25-26, if said HLA haplotype is A*6801.

In a further embodiment,
i. said at least one antigenic peptide comprises or consists of an epitope being selected from the group consisting of SEQ ID NOS: 27-37, if said HLA haplotype is B*0702;
ii. said at least one antigenic peptide comprises or consists of an epitope being selected from the group consisting of SEQ ID NOs: 38-43, if said HLA haplotype is B*0801;
iii. said at least one antigenic peptide comprises or consists of an epitope being SEQ ID NO: 44, if said HLA haplotype is B*1801;
iv. said at least one antigenic peptide comprises or consists of an epitope being selected from the group consisting of SEQ ID NOs: 45-46, if said HLA haplotype is B*3701;
v. said at least one antigenic peptide comprises or consists of an epitope being selected from the group consisting of SEQ ID NOs: 47-52, if said HLA haplotype is B*4402; and/or
vi. said at least one antigenic peptide comprises or consists of an epitope being selected from the group consisting of SEQ ID NOs: 53-57, if said HLA haplotype is B*5101.

In a further embodiment,
i. said at least one antigenic peptide comprises or consists of an epitope being SEQ ID NO: 58 if said HLA haplotype is C*0202;
ii. said at least one antigenic peptide comprises or consists of an epitope being selected from the group consisting of SEQ ID NOs: 59-60 if said HLA haplotype is C*0304;
iii. said at least one antigenic peptide comprises or consists of an epitope being selected from the group consisting of SEQ ID NOs: 61-66 if said HLA haplotype is C*0401;
iv. said at least one antigenic peptide comprises or consists of an epitope being selected from the group consisting of SEQ ID NOs: 67-71 if said HLA haplotype is C*0501;
v. said at least one antigenic peptide comprises or consists of an epitope being selected from the group consisting of SEQ ID NOS: 72-73 if said HLA haplotype is C*0701; and/or
vi. said at least one antigenic peptide comprises or consists of an epitope being selected from the group consisting of SEQ ID NOS: 74-76 if said HLA haplotype is C*0702.

The ability of the pHLA to stimulate and expand T cells is different between the matched pairs of HLA and antigenic peptides. Thus, in a further embodiment,
i. said at least one antigenic peptide comprises or consists of an epitope being selected from the group consisting of SEQ ID NOs: 1-2, if said HLA haplotype is A*0101;
ii. said at least one antigenic peptide comprises or consists of an epitope being SEQ ID NO: 3, if said HLA haplotype is A*0201;
iii. said at least one antigenic peptide comprises or consists of an epitope being selected from the group consisting of SEQ ID NOs: 4-7, if said HLA haplotype is A*0301;
iv. said at least one antigenic peptide comprises or consists of an epitope being selected from the group consisting of SEQ ID NOS: 8-9, if said HLA haplotype is A*1101;
v. said at least one antigenic peptide comprises or consists of an epitope being SEQ ID NO: 11, if said HLA haplotype is A*2402;
vi. said at least one antigenic peptide comprises or consists of an epitope being selected from the group consisting of SEQ ID NOs: 22-23, if said HLA haplotype is A*2601;
vii. said at least one antigenic peptide comprises or consists of an epitope being SEQ ID NO: 24, if said HLA haplotype is A*3001; and/or
viii. said at least one antigenic peptide comprises or consists of an epitope being selected from the group consisting of SEQ ID NOS: 25-26, if said HLA haplotype is A*6801.

In a further embodiment,
i. said at least one antigenic peptide comprises or consists of an epitope being selected from the group consisting of SEQ ID NOS: 2, if said HLA haplotype is A*0101;
ii. said at least one antigenic peptide comprises or consists of an epitope being selected from the group consisting of SEQ ID NOs: 4-5 and 7, if said HLA haplotype is A*0301;
iii. said at least one antigenic peptide comprises or consists of an epitope being selected from the group consisting of SEQ ID NOS: 8-9, if said HLA haplotype is A*1101;
iv. said at least one antigenic peptide comprises or consists of an epitope being SEQ ID NO: 11, if said HLA haplotype is A*2402;
v. said at least one antigenic peptide comprises or consists of an epitope being selected from the group consisting of SEQ ID NOS: 22-23, if said HLA haplotype is A*2601;
vi. said at least one antigenic peptide comprises or consists of an epitope being SEQ ID NO: 24, if said HLA haplotype is A*3001; and/or
vii. said at least one antigenic peptide comprises or consists of an epitope being selected from the group consisting of SEQ ID NOs: 25-26, if said HLA haplotype is A*6801.

In a further embodiment,
i. said at least one antigenic peptide comprises or consists of an epitope being selected from the group consisting of SEQ ID NOS: 1-2, if said HLA haplotype is A*0101;
ii. said at least one antigenic peptide comprises or consists of an epitope being SEQ ID NO: 3, if said HLA haplotype is A*0201,;
iii. said at least one antigenic peptide comprises or consists of an epitope being selected from the group consisting of SEQ ID NOS: 4-7, if said HLA haplotype is A*0301;
iv. said at least one antigenic peptide comprises or consists of an epitope being selected from the group consisting of SEQ ID NOS: 8-9, if said HLA haplotype is A*1101;
v. said at least one antigenic peptide comprises or consists of an epitope being selected from the group consisting of SEQ ID NOs: 10-11 and 13, if said HLA haplotype is A*2402;
vi. said at least one antigenic peptide comprises or consists of an epitope being selected from the group consisting of SEQ ID NOs: 22-23, if said HLA haplotype is A*2601;
vii. said at least one antigenic peptide comprises or consists of an epitope being SEQ ID NO: 24, if said HLA haplotype is A*3001; and/or
viii. said at least one antigenic peptide comprises or consists of an epitope being selected from the group consisting of SEQ ID NOs: 25-26, if said HLA haplotype is A*6801.

In a further embodiment,
i. said at least one antigenic peptide comprises or consists of an epitope being selected from the group consisting of SEQ ID NOS: 1-2, if said HLA haplotype is A*0101;
ii. said at least one antigenic peptide comprises or consists of an epitope being SEQ ID NO: 3, if said HLA haplotype is A*0201;
iii. said at least one antigenic peptide comprises or consists of an epitope being selected from the group consisting of SEQ ID NOS: 4-7, if said HLA haplotype is A*0301;
iv. said at least one antigenic peptide comprises or consists of an epitope being selected from the group consisting of SEQ ID NOs: 8-9, if said HLA haplotype is A*1101;
v. said at least one antigenic peptide comprises or consists of an epitope being selected from the group consisting of SEQ ID NOs: 10-11, 13, 16 and 20, if said HLA haplotype is A*2402;
vi. said at least one antigenic peptide comprises or consists of an epitope being selected from the group consisting of SEQ ID NOs: 22-23, if said HLA haplotype is A*2601;
vii. said at least one antigenic peptide comprises or consists of an epitope being SEQ ID NO: 24, if said HLA haplotype is A*3001; and/or
viii. said at least one antigenic peptide comprises or consists of an epitope being selected from the group consisting of SEQ ID NOS: 25-26, if said HLA haplotype is A*6801.

In a further embodiment,
i. said at least one antigenic peptide comprises or consists of an epitope being SEQ ID NO: 35, if said HLA haplotype is B*0702;
ii. said at least one antigenic peptide comprises or consists of an epitope being selected from the group consisting of SEQ ID NOs: 38-40 and 42-43, if said HLA haplotype is B*0801;
iii. said at least one antigenic peptide comprises or consists of an epitope being SEQ ID NO: 44, if said HLA haplotype is B*1801;
iv. said at least one antigenic peptide comprises or consists of an epitope being SEQ ID NO: 46, if said HLA haplotype is B*3701;
v. said at least one antigenic peptide comprises or consists of an epitope being selected from the group consisting of SEQ ID NOs: 47-52, if said HLA haplotype is B*4402; and/or
vi. said at least one antigenic peptide comprises or consists of an epitope being selected from the group consisting of SEQ ID NOs: 53-57, if said HLA haplotype is B*5101.

In a further embodiment,
i. said at least one antigenic peptide comprises or consists of an epitope being SEQ ID NO: 35, if said HLA haplotype is B*0702;
ii. said at least one antigenic peptide comprises or consists of an epitope being selected from the group consisting of SEQ ID NOs: 38, 40 and 42-43, if said HLA haplotype is B*0801;
iii. said at least one antigenic peptide comprises or consists of an epitope being SEQ ID NO: 44, if said HLA haplotype is B*1801;
iv. said at least one antigenic peptide comprises or consists of an epitope being selected from the group consisting of SEQ ID NOs: 47-48 and 50-52, if said HLA haplotype is B*4402; and/or
v. said at least one antigenic peptide comprises or consists of an epitope being selected from the group consisting of SEQ ID NOs: 53-57, if said HLA haplotype is B*5101.

In a further embodiment,
i. said at least one antigenic peptide comprises or consists of an epitope being selected from the group consisting of SEQ ID NOs: 32 and 35, if said HLA haplotype is B*0702;
ii. said at least one antigenic peptide comprises or consists of an epitope being selected from the group consisting of SEQ ID NOS: 38-43, if said HLA haplotype is B*0801;
iii. said at least one antigenic peptide comprises or consists of an epitope being SEQ ID NO: 44, if said HLA haplotype is B*1801;
iv. said at least one antigenic peptide comprises or consists of an epitope being selected from the group consisting of SEQ ID NOs: 45-46, if said HLA haplotype is B*3701;
v. said at least one antigenic peptide comprises or consists of an epitope being selected from the group consisting of SEQ ID NOs: 47-52, if said HLA haplotype is B*4402; and/or
vi. said at least one antigenic peptide comprises or consists of an epitope being selected from the group consisting of SEQ ID NOs: 53-57, if said HLA haplotype is B*5101.

In a further embodiment,
i. said at least one antigenic peptide comprises or consists of an epitope being selected from the group consisting of SEQ ID NOS: 27, 30-33 and 35-36, if said HLA haplotype is B*0702;
ii. said at least one antigenic peptide comprises or consists of an epitope being selected from the group consisting of SEQ ID NOs: 38-43, if said HLA haplotype is B*0801;
iii. said at least one antigenic peptide comprises or consists of an epitope being SEQ ID NO: 44, if said HLA haplotype is B*1801;
iv. said at least one antigenic peptide comprises or consists of an epitope being selected from the group consisting of SEQ ID NOs: 45-46, if said HLA haplotype is B*3701;
v. said at least one antigenic peptide comprises or consists of an epitope being selected from the group consisting of SEQ ID NOs: 47-52, if said HLA haplotype is B*4402; and/or
vi. said at least one antigenic peptide comprises or consists of an epitope being selected from the group consisting of SEQ ID NOs: 53-57, if said HLA haplotype is B*5101.

In a further embodiment,
i. said at least one antigenic peptide comprises or consists of an epitope being SEQ ID NO: 58, if said HLA haplotype is C*0202;
ii. said at least one antigenic peptide comprises or consists of an epitope being SEQ ID NO: 59, if said HLA haplotype is C*0304;
iii. said at least one antigenic peptide comprises or consists of an epitope being SEQ ID NO: 62, if said HLA haplotype is C*0401;
iv. said at least one antigenic peptide comprises or consists of an epitope being SEQ ID NO: 68, if said HLA haplotype is C*0501;
v. said at least one antigenic peptide comprises or consists of an epitope being selected from the group consisting of SEQ ID NOs: 72-73, if said HLA haplotype is C*0701; and/or
vi. said at least one antigenic peptide comprises or consists of an epitope being SEQ ID NO: 75, if said HLA haplotype is C*0702.

In a further embodiment,
i. said at least one antigenic peptide comprises or consists of an epitope being SEQ ID NO: 58 if said HLA haplotype is C*0202;
ii. said at least one antigenic peptide comprises or consists of an epitope being selected from the group consisting of SEQ ID NOs: 59-60, if said HLA haplotype is C*0304;
iii. said at least one antigenic peptide comprises or consists of an epitope being selected from the group consisting of SEQ ID NOs: 61-66, if said HLA haplotype is C*0401;
iv. said at least one antigenic peptide comprises or consists of an epitope being selected from the group consisting of SEQ ID NOs: 68-69, if said HLA haplotype is C*0501;
v. said at least one antigenic peptide comprises or consists of an epitope being selected from the group consisting of SEQ ID NOS: 72-73, if said HLA haplotype is C*0701; and/or
vi. said at least one antigenic peptide comprises or consists of an epitope being selected from the group consisting of SEQ ID NOs: 74-76, if said HLA haplotype is C*0702.

In a further embodiment,
i. said at least one antigenic peptide comprises or consists of an epitope being SEQ ID NO: 58, if said HLA haplotype is C*0202;
ii. said at least one antigenic peptide comprises or consists of an epitope being selected from the group consisting of SEQ ID NOs: 59-60, if said HLA haplotype is C*0304;
iii. said at least one antigenic peptide comprises or consists of an epitope being selected from the group consisting of SEQ ID NOs: 61-66, if said HLA haplotype is C*0401;
iv. said at least one antigenic peptide comprises or consists of an epitope being selected from the group consisting of SEQ ID NOs: 67-71, if said HLA haplotype is C*0501;
v. said at least one antigenic peptide comprises or consists of an epitope being selected from the group consisting of SEQ ID NOs: 72-73, if said HLA haplotype is C*0701; and/or
vi. said at least one antigenic peptide comprises or consists of an epitope being selected from the group consisting of SEQ ID NOs: 74-76, if said HLA haplotype is C*0702.

One embodiment of the present invention as described herein relates to said APC comprising at least one HLA haplotype being A*0101 and at least one antigenic peptide comprising or consisting of an epitope according to SEQ ID NO: 1.

A further embodiment of the present invention as described herein relates to said APC comprising at least one HLA haplotype being A*0101 and at least one antigenic peptide comprising or consisting of an epitope according to SEQ ID NO: 2.

A further embodiment of the present invention as described herein relates to said APC comprising at least one HLA haplotype being A*0201 and at least one antigenic peptide comprising or consisting of an epitope according to SEQ ID NO: 3.

A further embodiment of the present invention as described herein relates to said APC comprising at least one HLA haplotype being A*0301 and at least one antigenic peptide comprising or consisting of an epitope according to SEQ ID NO: 4.

A further embodiment of the present invention as described herein relates to said APC comprising at least one HLA haplotype being A*0301 and at least one antigenic peptide comprising or consisting of an epitope according to SEQ ID NO: 5.

A further embodiment of the present invention as described herein relates to said APC comprising at least one HLA haplotype being A*0301 and at least one antigenic peptide comprising or consisting of an epitope according to SEQ ID NO: 6.

A further embodiment of the present invention as described herein relates to said APC comprising at least one HLA haplotype being A*0301 and at least one antigenic peptide comprising or consisting of an epitope according to SEQ ID NO: 7.

A further embodiment of the present invention as described herein relates to said APC comprising at least one HLA haplotype being A*1101 and at least one antigenic peptide comprising or consisting of an epitope according to SEQ ID NO: 8.

A further embodiment of the present invention as described herein relates to said APC comprising at least one HLA haplotype being A*1101 and at least one antigenic peptide comprising or consisting of an epitope according to SEQ ID NO: 9.

A further embodiment of the present invention as described herein relates to said APC comprising at least one HLA haplotype being A*2402 and at least one antigenic peptide comprising or consisting of an epitope according to SEQ ID NO: 10.

A further embodiment of the present invention as described herein relates to said APC comprising at least one HLA haplotype being A*2402 and at least one antigenic peptide comprising or consisting of an epitope according to SEQ ID NO: 11.

A further embodiment of the present invention as described herein relates to said APC comprising at least one HLA haplotype being A*2402 and at least one antigenic peptide comprising or consisting of an epitope according to SEQ ID NO: 12.

A further embodiment of the present invention as described herein relates to said APC comprising at least one HLA haplotype being A*2402 and at least one antigenic peptide comprising or consisting of an epitope according to SEQ ID NO: 13.

A further embodiment of the present invention as described herein relates to said APC comprising at least one HLA haplotype being A*2402 and at least one antigenic peptide comprising or consisting of an epitope according to SEQ ID NO: 14.

A further embodiment of the present invention as described herein relates to said APC comprising at least one HLA haplotype being A*2402 and at least one antigenic peptide comprising or consisting of an epitope according to SEQ ID NO: 15.

A further embodiment of the present invention as described herein relates to said APC comprising at least one HLA haplotype being A*2402 and at least one antigenic peptide comprising or consisting of an epitope according to SEQ ID NO: 16.

A further embodiment of the present invention as described herein relates to said APC comprising at least one HLA haplotype being A*2402 and at least one antigenic peptide comprising or consisting of an epitope according to SEQ ID NO: 17.

A further embodiment of the present invention as described herein relates to said APC comprising at least one HLA haplotype being A*2402 and at least one antigenic peptide comprising or consisting of an epitope according to SEQ ID NO: 18.

A further embodiment of the present invention as described herein relates to said APC comprising at least one HLA haplotype being A*2402 and at least one antigenic peptide comprising or consisting of an epitope according to SEQ ID NO: 19.

A further embodiment of the present invention as described herein relates to said APC comprising at least one HLA haplotype being A*2402 and at least one antigenic peptide comprising or consisting of an epitope according to SEQ ID NO: 20.

A further embodiment of the present invention as described herein relates to said APC comprising at least one HLA haplotype being A*2402 and at least one antigenic peptide comprising or consisting of an epitope according to SEQ ID NO: 21.

A further embodiment of the present invention as described herein relates to said APC comprising at least one HLA haplotype being A*2601 and at least one antigenic peptide comprising or consisting of an epitope according to SEQ ID NO: 22.

A further embodiment of the present invention as described herein relates to said APC comprising at least one HLA haplotype being A*2601 and at least one antigenic peptide comprising or consisting of an epitope according to SEQ ID NO: 23.

A further embodiment of the present invention as described herein relates to said APC comprising at least one HLA haplotype being A*3001 and at least one antigenic peptide comprising or consisting of an epitope according to SEQ ID NO: 24.

A further embodiment of the present invention as described herein relates to said APC comprising at least one HLA haplotype being A*6801 and at least one antigenic peptide comprising or consisting of an epitope according to SEQ ID NO: 25.

A further embodiment of the present invention as described herein relates to said APC comprising at least one HLA haplotype being A*6801 and at least one antigenic peptide comprising or consisting of an epitope according to SEQ ID NO: 26.

A further embodiment of the present invention as described herein relates to said APC comprising at least one HLA haplotype being B*0702 and at least one antigenic peptide comprising or consisting of an epitope according to SEQ ID NO: 27.

A further embodiment of the present invention as described herein relates to said APC comprising at least one HLA haplotype being B*0702 and at least one antigenic peptide comprising or consisting of an epitope according to SEQ ID NO: 28.

A further embodiment of the present invention as described herein relates to said APC comprising at least one HLA haplotype being B*0702 and at least one antigenic peptide comprising or consisting of an epitope according to SEQ ID NO: 29.

A further embodiment of the present invention as described herein relates to said APC comprising at least one HLA haplotype being B*0702 and at least one antigenic peptide comprising or consisting of an epitope according to SEQ ID NO: 30.

A further embodiment of the present invention as described herein relates to said APC comprising at least one HLA haplotype being B*0702 and at least one antigenic peptide comprising or consisting of an epitope according to SEQ ID NO: 31.

A further embodiment of the present invention as described herein relates to said APC comprising at least one HLA haplotype being B*0702 and at least one antigenic peptide comprising or consisting of an epitope according to SEQ ID NO: 32.

A further embodiment of the present invention as described herein relates to said APC comprising at least one HLA haplotype being B*0702 and at least one antigenic peptide comprising or consisting of an epitope according to SEQ ID NO: 33.

A further embodiment of the present invention as described herein relates to said APC comprising at least one HLA haplotype being B*0702 and at least one antigenic peptide comprising or consisting of an epitope according to SEQ ID NO: 34.

A further embodiment of the present invention as described herein relates to said APC comprising at least one HLA haplotype being B*0702 and at least one antigenic peptide comprising or consisting of an epitope according to SEQ ID NO: 35.

A further embodiment of the present invention as described herein relates to said APC comprising at least one HLA haplotype being B*0702 and at least one antigenic peptide comprising or consisting of an epitope according to SEQ ID NO: 36.

A further embodiment of the present invention as described herein relates to said APC comprising at least one HLA haplotype being B*0702 and at least one antigenic peptide comprising or consisting of an epitope according to SEQ ID NO: 37.

A further embodiment of the present invention as described herein relates to said APC comprising at least one HLA haplotype being B*0801 and at least one antigenic peptide comprising or consisting of an epitope according to SEQ ID NO: 38.

A further embodiment of the present invention as described herein relates to said APC comprising at least one HLA haplotype being B*0801 and at least one antigenic peptide comprising or consisting of an epitope according to SEQ ID NO: 39.

A further embodiment of the present invention as described herein relates to said APC comprising at least one HLA haplotype being B*0801 and at least one antigenic peptide comprising or consisting of an epitope according to SEQ ID NO: 40.

A further embodiment of the present invention as described herein relates to said APC comprising at least one HLA haplotype being B*0801 and at least one antigenic peptide comprising or consisting of an epitope according to SEQ ID NO: 41.

A further embodiment of the present invention as described herein relates to said APC comprising at least one HLA haplotype being B*0801 and at least one antigenic peptide comprising or consisting of an epitope according to SEQ ID NO: 42.

A further embodiment of the present invention as described herein relates to said APC comprising at least one HLA haplotype being B*0801 and at least one antigenic peptide comprising or consisting of an epitope according to SEQ ID NO: 43.

A further embodiment of the present invention as described herein relates to said APC comprising at least one HLA haplotype being B*1801 and at least one antigenic peptide comprising or consisting of an epitope according to SEQ ID NO: 44.

A further embodiment of the present invention as described herein relates to said APC comprising at least one HLA haplotype being B*3701 and at least one antigenic peptide comprising or consisting of an epitope according to SEQ ID NO: 45.

A further embodiment of the present invention as described herein relates to said APC comprising at least one HLA haplotype being B*3701 and at least one antigenic peptide comprising or consisting of an epitope according to SEQ ID NO: 46.

A further embodiment of the present invention as described herein relates to said APC comprising at least one HLA haplotype being B*4402 and at least one antigenic peptide comprising or consisting of an epitope according to SEQ ID NO: 47.

A further embodiment of the present invention as described herein relates to said APC comprising at least one HLA haplotype being B*4402 and at least one antigenic peptide comprising or consisting of an epitope according to SEQ ID NO: 48.

A further embodiment of the present invention as described herein relates to said APC comprising at least one HLA haplotype being B*4402 and at least one antigenic peptide comprising or consisting of an epitope according to SEQ ID NO: 49.

A further embodiment of the present invention as described herein relates to said APC comprising at least one HLA haplotype being B*4402 and at least one antigenic peptide comprising or consisting of an epitope according to SEQ ID NO: 50.

A further embodiment of the present invention as described herein relates to said APC comprising at least one HLA haplotype being B*4402 and at least one antigenic peptide comprising or consisting of an epitope according to SEQ ID NO: 51.

A further embodiment of the present invention as described herein relates to said APC comprising at least one HLA haplotype being B*4402 and at least one antigenic peptide comprising or consisting of an epitope according to SEQ ID NO: 52.

A further embodiment of the present invention as described herein relates to said APC comprising at least one HLA haplotype being B*5101 and at least one antigenic peptide comprising or consisting of an epitope according to SEQ ID NO: 53.

A further embodiment of the present invention as described herein relates to said APC comprising at least one HLA haplotype being B*5101 and at least one antigenic peptide comprising or consisting of an epitope according to SEQ ID NO: 54.

A further embodiment of the present invention as described herein relates to said APC comprising at least one HLA haplotype being B*5101 and at least one antigenic peptide comprising or consisting of an epitope according to SEQ ID NO: 55.

A further embodiment of the present invention as described herein relates to said APC comprising at least one HLA haplotype being B*5101 and at least one antigenic peptide comprising or consisting of an epitope according to SEQ ID NO: 56.

A further embodiment of the present invention as described herein relates to said APC comprising at least one HLA haplotype being B*5101 and at least one antigenic peptide comprising or consisting of an epitope according to SEQ ID NO: 57.

A further embodiment of the present invention as described herein relates to said APC comprising at least one HLA haplotype being C*0202 and at least one antigenic peptide comprising or consisting of an epitope according to SEQ ID NO: 58.

A further embodiment of the present invention as described herein relates to said APC comprising at least one HLA haplotype being C*0304 and at least one antigenic peptide comprising or consisting of an epitope according to SEQ ID NO: 59.

A further embodiment of the present invention as described herein relates to said APC comprising at least one HLA haplotype being C*0304 and at least one antigenic peptide comprising or consisting of an epitope according to SEQ ID NO: 60.

A further embodiment of the present invention as described herein relates to said APC comprising at least one HLA haplotype being C*0401 and at least one antigenic peptide comprising or consisting of an epitope according to SEQ ID NO: 61.

A further embodiment of the present invention as described herein relates to said APC comprising at least one HLA haplotype being C*0401 and at least one antigenic peptide comprising or consisting of an epitope according to SEQ ID NO: 62.

A further embodiment of the present invention as described herein relates to said APC comprising at least one HLA haplotype being C*0401 and at least one antigenic peptide comprising or consisting of an epitope according to SEQ ID NO: 63.

A further embodiment of the present invention as described herein relates to said APC comprising at least one HLA haplotype being C*0401 and at least one antigenic peptide comprising or consisting of an epitope according to SEQ ID NO: 64.

A further embodiment of the present invention as described herein relates to said APC comprising at least one HLA haplotype being C*0401 and at least one antigenic peptide comprising or consisting of an epitope according to SEQ ID NO: 65.

A further embodiment of the present invention as described herein relates to said APC comprising at least one HLA haplotype being C*0401 and at least one antigenic peptide comprising or consisting of an epitope according to SEQ ID NO: 66.

A further embodiment of the present invention as described herein relates to said APC comprising at least one HLA haplotype being C*0501 and at least one antigenic peptide comprising or consisting of an epitope according to SEQ ID NO: 67.

A further embodiment of the present invention as described herein relates to said APC comprising at least one HLA haplotype being C*0501 and at least one antigenic peptide comprising or consisting of an epitope according to SEQ ID NO: 68.

A further embodiment of the present invention as described herein relates to said APC comprising at least one HLA haplotype being C*0501 and at least one antigenic peptide comprising or consisting of an epitope according to SEQ ID NO: 69.

A further embodiment of the present invention as described herein relates to said APC comprising at least one HLA haplotype being C*0501 and at least one antigenic peptide comprising or consisting of an epitope according to SEQ ID NO: 70.

A further embodiment of the present invention as described herein relates to said APC comprising at least one HLA haplotype being C*0501 and at least one antigenic peptide comprising or consisting of an epitope according to SEQ ID NO: 71.

A further embodiment of the present invention as described herein relates to said APC comprising at least one HLA haplotype being C*0701 and at least one antigenic peptide comprising or consisting of an epitope according to SEQ ID NO: 72.

A further embodiment of the present invention as described herein relates to said APC comprising at least one HLA haplotype being C*0701 and at least one antigenic peptide comprising or consisting of an epitope according to SEQ ID NO: 73.

A further embodiment of the present invention as described herein relates to said APC comprising at least one HLA haplotype being C*0702 and at least one antigenic peptide comprising or consisting of an epitope according to SEQ ID NO: 74.

A further embodiment of the present invention as described herein relates to said APC comprising at least one HLA haplotype being C*0702 and at least one antigenic peptide comprising or consisting of an epitope according to SEQ ID NO: 75.

A further embodiment of the present invention as described herein relates to said APC comprising at least one HLA haplotype being C*0702 and at least one antigenic peptide comprising or consisting of an epitope according to SEQ ID NO: 76.

### Samples

In order to be able to expand a person-tailored T cell composition, it is needed that the HLA of the subject for which the T cell composition is person-tailored is known. Thus, at least one identified HLA haplotype of the subject is to be provided. In one embodiment, said at least one identified HLA haplotype is identified by providing a biological sample from said subject and identifying at least one HLA haplotype from said biological sample.

Alternatively, it may be provided by already being available e.g. if it is already known to the subject or if it is available in the medical record of the subject.

If a biological sample is to be provided for identifying the HLA haplotype of the subject, it can be obtained from easily accessible tissues such as blood, buckle swap, biopsies, archival tissue (from pathologist), tumor, lymphoid system.

The biological sample may be from a different tissue than the sample comprising T cells, which is used as the source for expansion of the T cells or the biological sample and the sample comprising T cells may be from the same tissue. In one embodiment, said biological sample is identical to said sample comprising T cells. In a further embodiment, said biological sample and said sample comprising T cells are from the same subject.

The sample comprising the T cells to be expanded may originate from any source, but is typically extracted from blood, a tissue or a body fluid. Thus, in one embodiment, said sample comprising T cells is selected from the group consisting of peripheral blood mononuclear cells, tumors, tissue, bone marrow, biopsies, serum, blood, plasma, saliva, lymph fluid, pleura fluid, cerospinal fluid and synovial fluid.

The sample comprising the T cells to be expanded according to the method described herein may also be selected from stem cells or TCR modified/transduced cells.

The sample comprising the T cells is extracted from a subject and subsequently put into a culture comprising the APC under conditions that allow growth of the T cells. Thus, it is to be understood that the expansion of the T cells is to be carried out in a solution or medium that in addition to the APC contains all the necessary compounds and factors for cell proliferation. Thus, the culture in which the T cell expansion is carried out may contain compounds that inhibit growth of irrelevant cells or promote growth of the T cells, e.g. IL-2.

In one embodiment, the T cells are CD8+ T cells.

For the re-introduction of an expanded T cell population into a patient to be meaningful from a therapeutic perspective, it is necessary that the extracted T cells are expanded to a clinically relevant number. Expansion of T cells by the method of the present invention may be on the order of 50-3000 fold. The number of cells available before re-introduction into a patient may be in the range of 10⁴-10¹² cells per administration, such as 10⁵-10¹⁰ cells per administration, such as 10⁶-10⁹ cells per administration. Cells are administered in a volume of 20 mL to 1 L depending on the route of administration.

Thus, an embodiment of the present invention relates to the method as described herein, wherein the T cells are expanded to a clinically relevant number.

### APC

In order to expand a person-tailored T cell composition, an antigen presenting cell is needed (APC), which may present the pHLA of relevance. The APC could be any natural or artificial cell capable of presenting the pHLA to the T cells in the sample comprising T cells when in contact herewith. One embodiment of the present invention relates to a method wherein said APC is an artificial APC (aAPC), an autologous APC or an allogenic APC.

Autologous APCs and allogenic APCs would interact with T cells, driven by the TCR, antigenic peptide/HLA interaction. Hereby, T cells would be provided with additional growth and activation stimuli based on the formation of an immunological synapse (cluster of signalling molecules). The natural APCs would be generated to present the antigenic peptides as described herein with the matched HLA haplotypes.

An aAPC is an APC which is artificially created in order to enable pHLA to be presented. The simple concept behind aAPCs is that they mimic the natural interaction between the TCR and the specific antigenic peptide presented by the major histocompatibility complex (MHC). A further embodiment of the present invention as described herein relates to a method, wherein said aAPC is selected from the group consisting of an aAPC scaffold, aAPC beads or a cellular aAPC.

In one embodiment, said aAPC is an aAPC scaffold. An aAPC scaffold may be designed in different ways but may typically include a backbone to which the pHLA is connected. It is to be understood that the HLA may be attached to the aAPC scaffold without the antigenic peptide. The antigenic peptide may be added prior to use, matching relevant HLA with relevant antigenic peptide.

The backbone may be a polymeric backbone. The polymeric backbone may be of a material selected from polysaccharides, dextranes, vinyl polymers, poly ethylene glycol, poly propylene glycol, strep-tactin, poly-streptavidin, biotin-binding proteins and polyhistidine-binding polymers. Examples of aAPCs are described in WO2018115146 and WO2019243463, which are hereby incorporated by reference.

The aAPCs may also comprise other molecules attached to the backbone besides the pHLA in order to stimulate the expansion of the T cells such as T cell affecting molecules. Thus, one embodiment of the present invention relates to a method wherein said aAPC scaffold comprises at least one gamma-chain receptor cytokine such as IL-21, IL-2, IL-15, IL-4, IL-9 and IL-7. In a further embodiment, said aAPC scaffold comprises IL-2 and IL-21. In a still further embodiment, said aAPC scaffold comprises IL-15, IL-21 and IL-2. In a still further embodiment, said aAPC scaffold comprises IL-15 and IL-21.

In another embodiment, the aAPC scaffold comprises at least one co-stimulatory molecule such as B7.2 (CD86), B7.1 (CD80), CD40, ICOS and PD-L1.

In one embodiment, the aAPC scaffold comprises a dextran backbone being assembled with IL-2, IL-21 and MHC. In a further embodiment, the ratio between dextran backbone, the MHC, IL-2 and IL-21 is 1:24:6:6. In an even further embodiment, the ratio between dextran backbone, IL-2, IL-21, IL-15 and the MHC, is 1:18:6:6:6. In a still further embodiment, the ratio between dextran backbone, IL-2, IL-21, IL-15 and the MHC, is 1:24:6:6:6.

In one embodiment, the aAPC scaffold comprises a dextran backbone being assembled with neoIL-2/IL-15 (Silva et al., 2019) and MHC. In a further embodiment, the ratio between dextran backbone, neoIL2/IL15 and the MHC, is 1:24:3.

aAPC beads standardly comprises a bead to which a pHLA can be attached potentially together with other stimulatory molecules as described above for the aAPC scaffold. Bead-based aAPCs are commonly known to the skilled person in the art and examples of aAPC beads may be MACS Microbeads and Dynabeads as described e.g. in Ichikawa J et al, 2020, which is hereby incorporated by reference.

Cellular aAPC may be primary or transformed human or xenogeneic cells being engineered to express the relevant pHLA. Examples of cellular aAPC may be K562-HLA transduced cells, which is a GMP grade cell line for T cell stimulation.

In one embodiment, each APC may comprise one or more HLA haplotypes for expanding the T cells such as one HLA haplotype, two HLAs haplotypes or three HLAs haplotypes. Preferably, each APC only comprises one HLA haplotype. The number of HLA of the same haplotype on each APC could range from 1-100 depending on the type of APC used. In one embodiment, each APC comprises at least five HLA molecules of the same type.

In a further embodiment, each APC may comprise one or more pHLA haplotypes for expanding the T cells such as one pHLA haplotype, two pHLAs haplotypes or three pHLAs haplotypes. Preferably, each APC only comprises one pHLA haplotype. The number of pHLA of the same haplotype on each APC could range from 1-100 depending on the type of APC used. In one embodiment, each APC comprises at least five pHLA molecules of the same type.

In a further embodiment, the solution may comprise one or more different APCs having different pHLAs, where the HLA haplotype is similar but the antigenic peptide is different.

In a still further embodiment, the solution comprises three APCs being A*2402 matched with the antigenic peptide according to SEQ ID NO: 10; A*2402 matched with the antigenic peptide according to SEQ ID NO: 12 and A*2402 matched with the antigenic peptide according to SEQ ID NO: 13.

In an even further embodiment, the solution comprises three APCs being A*0201 matched with antigenic peptide according to SEQ ID NO: 3, A*2402 matched with antigenic peptide according to SEQ ID NO: 13 and B*4402 matched with antigenic peptide according to SEQ ID NO: 47.

The number of APC having different pHLAs i.e. the number of different APCs used for the expansion of the T cells can be one or more. In one embodiment, the solution comprises at least fifty different APCs, such as at least fourty-five different APCs, like at least fourty different APCs, such as at least thirty-five different APCs, like at least thirty different APCs, such as at least twenty-five different APCs, like at least twenty different APCs, such as at least eighteen different APCs, like at least sixteen different APCs, such as at least fourteen different APCs, like at least twelve different APCs, such as at least ten different APCs, like at least eight different APCs, such as at least six different APCs, like at least four different APCs, such as at least three different APCs.

A further embodiment of the present invention relates to a method wherein the solution comprises a number of different APCs resembling the number of identified HLA haplotypes. Hereby, a person-tailored T cell composition, which includes expanded T cells for all of the HLA haplotypes identified in the subject to whom the T cell composition has been person-tailored, would be obtained. T cells thus have been expanded against several epitopes of the subject corresponding to each of the identified HLA haplotypes.

In a further embodiment, the solution comprises a number of different APCs being at least twice the number of identified HLA haplotypes, where the different APCs comprises different pHLAs. In an even further embodiment, the solution comprises a number of different APCs being at least three-times the number of identified HLA haplotypes, where the different APCs comprises different pHLAs.

### Sorting Step

In order to obtain a more efficient person-tailored T cell composition, the T cells may be sorted before they are expanded. This enables only the T cells of interest to be expanded and irrelevant T cells, which may be in the composition “polluting” the composition. Hence, one embodiment of the present invention as described herein relates to a method, wherein the method further comprises the step of separating or sorting the T cells prior to expanding them.

The step of sorting the stimulated T cells may be performed by using labelled antigenic peptides, labelled pHLAs or labelled APCs bearing the relevant pHLA. Hereby, the stimulated T cells of relevance will bind to the labelled antigenic peptide, labelled pHLAs or labelled APCs under conditions promoting binding. Depending on the specific label, the sorting may be performed by e.g. magnetism, fluorescence or microfluidics. In one embodiment, the T cells are separated by magnetic separation. In a further embodiment, the T cells are sorted by fluorescence sorting.

### Use of Person-Tailored T Cell Composition

It is envisioned that the expanded T cell population obtained by the method of the present invention can be used effectively in a treatment regimen focusing on adoptive immunotherapy (or adoptive cell transfer). In such a treatment regimen, T cells from a subject in need of treatment are extracted. With respect to the present invention as described herein, the subject is a human. Thus, an aspect of the present invention relates to a person-tailored T cell composition obtained by a method as described herein.

The T cell composition as described herein have several favourable characteristics such as a high fraction of antigen specific cells designed directly to the subject in need of treatment.

The person-tailored T cell composition obtained by the method as described herein may be formulated in a pharmaceutical composition further comprising one or more adjuvants and/or excipients and/or a pharmaceutically acceptable carrier. The excipients may include, but are not limited to, buffers, suspending agents, dispersing agents, solubilising agents, pH-adjusting agents and/or preserving agents.

The pharmaceutical composition may be used in adoptive immunotherapy (or adoptive cell transfer) for administration either locally or systemically via any route, such as intravenous, intraperitoneal, intramuscular, subcutaneous, transdermal or oral.

A further aspect of the present invention relates to a person-tailored T cell composition as described herein for use as a medicament.

It should be noted that the antigenic peptides used in the method for obtaining the person-tailored T cell composition are derived from Merkel Cell polyomavirus, and in particular from the LTA, STA and CT genes of the Merkel Cell polyomavirus. Accordingly, the person-tailored T cell composition would hold T cells capable of recognising epitopes on cells affected by Merkel Cell polyomavirus.

A further aspect of the present invention relates to a person-tailored T cell composition as described herein for use in the prevention, alleviation and/or treatment of Merkel Cell Carcinoma.

Accordingly, the person-tailored T cell composition as described herein can be used for the treatment of a subject suffering from Merkel Cell Carcinoma, where the T cell composition is directed towards the specific pHLA haplotypes of the subject to be treated.

Thus, a further aspect of the present invention relates to a method of treating a Merkel Cell Carcinoma in a subject comprising administering to the subject a person-tailored T cell composition as prepared by the method as described herein.

### Kit for Expansion of a T Cell Composition

The APC having HLA of a given haplotype may be part of a kit suitable for use by hospitals and laboratories. Such a kit may comprise one or more different APC suitable for expanding T cells with different specificities together with matched antigenic peptides for combining with the corresponding HLA on the APC. The kit may furthermore comprise a medium suitable for expanding T cells in a sample comprising T cells as well as other compounds or molecules necessary for the expansion of a person-tailored T cell composition.

Thus, an aspect of the present invention relates to a

kit in parts for expansion of T cells, the kit comprising:
i) at least one APC having a given HLA haplotype;
ii) at least one antigenic peptide wherein said antigenic peptide comprises an epitope from Merkel cell polyomavirus, said epitope originates from large T antigen (LTA), small T antigen (STA) or the shared region (CT) of LTA and STA; and
   wherein said at least one APC and said at least one antigenic peptide are configured to be combined by combining said given HLA haplotype with a matched antigenic peptide.

In one embodiment, said APC is an aAPC scaffold. In one embodiment, the aAPC scaffold comprises IL-2 and IL-21. In a further embodiment, the aAPC scaffold comprises IL-15, IL-21 and IL-2. In a still further embodiment, the aAPC scaffold comprises IL-15 and IL-21.

In one embodiment, the aAPC scaffold comprises a dextran backbone being assembled with IL-2, IL-21 and MHC. In a further embodiment, the ratio between dextran backbone, the MHC, IL-2 and IL-21 is 1:24:6:6. In an even further embodiment, the ratio between dextran backbone, IL-2, IL-21, IL-15 and the MHC, is 1:18:6:6:6. In a still further embodiment, the ratio between dextran backbone, IL-2, IL-21, IL-15 and the MHC, is 1:24:6:6:6.

In one embodiment, the aAPC scaffold comprises a dextran backbone being assembled with neoIL-2/IL-15 (Silva et al., 2019) and MHC. In a further embodiment, the ratio between dextran backbone, neoIL2/IL15 and the MHC, is 1:24:3.

An embodiment of the present invention relates to the kit as described herein, comprising a library of APCs with different HLA haplotypes. The library of APCs may contain a selection of the most frequently used APCs such as the APCs with the HLA haplotypes most commonly observed in a specific geographic area. In one embodiment, the library of APCs comprises the following HLA haplotypes: A*101, A*0201, A*0301, A*1101, A*2402, A*2601, A*3001, A*6801, B*0702, B*0801, B*1801, B*3701, B*4402, B*5101, C*0202, C*0304, C*0401, C*0501, C*0701 and C*0702.

An embodiment of the present invention relates to the kit as described herein, comprising a library of antigenic peptides. The library of antigenic peptides may contain a selection of the most frequently used antigenic peptides such as the antigenic peptides corresponding to the HLA haplotypes most commonly observed in a specific geographic area. In one embodiment, the library of antigenic peptides comprises or consists of epitopes according to SEQ ID NOS: 1-76. In a further embodiment, the library of antigenic peptides comprises or consists of epitopes according to SEQ ID NOs: 2, 4-12, 14-26, 29-38, 40-45, 47-48 and 50-76.

In a further embodiment, the kit comprises a first storage means being a library of APCs as described herein and a second storage means being a library of antigenic peptides as described herein. The storage means may be containers for storing the individual APCs and the individual antigenic peptides.

A kit would enable onsite stimulation and expansion of T cells to obtain a person-tailored T cell composition. For example, a subject is diagnosed at a hospital with Merkel Cell Carcinoma. The HLA haplotype of the subject is provided from a biological sample. Specific APCs having HLA haplotypes corresponding to the provided HLA haplotypes can be identified from the library. Hereafter, antigenic peptides matched to the specific HLA haplotypes can be chosen. One antigenic peptide, two antigenic peptides, three antigenic peptides or more can be selected per HLA haplotype depending on the number of antigenic peptides matched per the specific HLA haplotype and wishes to the final composition. The antigenic peptides are then combined with the matched HLA haplotypes on the APCs and relevant T cells can be obtained by expansion and stimulation.

Alternatively, a first combination can be chosen for a first person-tailored T cell composition and at a later point in time a second combination could be chosen for a second person-tailored T cell composition. For example if sufficient effect is not obtained by the T cell composition obtained by the first combination.

Accordingly, one embodiment of the present invention relates to a kit as described herein, wherein
i. said matched antigenic peptide comprises or consists of an epitope being selected from the group consisting of SEQ ID NOS: 1-2, if said given HLA haplotype is A*0101;
ii. said matched antigenic peptide comprises or consists of an epitope being SEQ ID NO: 3, if said given HLA haplotype is A*0201;
iii. said matched antigenic peptide comprises or consists of an epitope being selected from the group consisting of SEQ ID NOs: 4-7, if said given HLA haplotype is A*0301;
iv. said matched antigenic peptide comprises or consists of an epitope being selected from the group consisting of SEQ ID NOS: 8-9, if said given HLA haplotype is A*1101;
v. said matched antigenic peptide comprises or consists of an epitope being selected from the group consisting of SEQ ID NOs: 10-21, if said given HLA haplotype is A*2402;
vi. said matched antigenic peptide comprises or consists of an epitope being selected from the group consisting of SEQ ID NOs: 22-23, if said given HLA haplotype is A*2601;
vii. said matched antigenic peptide comprises or consists of an epitope being SEQ ID NO: 24, if said given HLA haplotype is A*3001;
viii. said matched antigenic peptide comprises or consists of an epitope being selected from the group consisting of SEQ ID NOs: 25-26, if said given HLA haplotype is A*6801;
ix. said matched antigenic peptide comprises or consists of an epitope being selected from the group consisting of SEQ ID NOs: 27-37, if said given HLA haplotype is B*0702;
x. said matched antigenic peptide comprises or consists of an epitope being selected from the group consisting of SEQ ID NOs: 38-43, if said given HLA haplotype is B*0801;
xi. said matched antigenic peptide comprises or consists of an epitope being SEQ ID NO: 44, if said given HLA haplotype is B*1801;
xii. said matched antigenic peptide comprises or consists of an epitope being selected from the group consisting of SEQ ID NOs: 45-46, if said given HLA haplotype is B*3701;
xiii. said matched antigenic peptide comprises or consists of an epitope being selected from the group consisting of SEQ ID NOs: 47-52, if said given HLA haplotype is B*4402;
xiv. said matched antigenic peptide comprises or consists of an epitope being selected from the group consisting of SEQ ID NOs: 53-57, if said given HLA haplotype is B*5101;
xv. said matched antigenic peptide comprises or consists of an epitope being SEQ ID NO: 58 if said given HLA haplotype is C*0202;
xvi. said matched antigenic peptide comprises or consists of an epitope being selected from the group consisting of SEQ ID NOs: 59-60 if said given HLA haplotype is C*0304;
xvii. said matched antigenic peptide comprises or consists of an epitope being selected from the group consisting of SEQ ID NOs: 61-66 if said given HLA haplotype is C*0401;
xviii. said matched antigenic peptide is comprises or consists of an epitope being selected from the group consisting of SEQ ID NOs: 67-71 if said given HLA haplotype is C*0501;
xix. said matched antigenic peptide comprises or consists of an epitope being selected from the group consisting of SEQ ID NOS: 72-73 if said given HLA haplotype is C*0701; and/or
xx. said matched antigenic peptide comprises or consists of an epitope being selected from the group consisting of SEQ ID NOs: 74-76 if said given HLA haplotype is C*0702.

A still further embodiment of the present invention relates to a kit as described herein, wherein
i. said matched antigenic peptide comprises or consists of an epitope being SEQ ID NO: 2, if said given HLA haplotype is A*0101;
ii. said matched antigenic peptide comprises or consists of an epitope being selected from the group consisting of SEQ ID NOs: 4-5 and SEQ ID NO: 7, if said given HLA haplotype is A*0301;
iii. said matched antigenic peptide comprises or consists of an epitope being selected from the group consisting of SEQ ID NOS: 8-9, if said given HLA haplotype is A*1101;
iv. said matched antigenic peptide comprises or consists of an epitope being selected from the group consisting of SEQ ID NOs: 10-12 and SEQ ID NO: 14-21, if said given HLA haplotype is A*2402;
v. said matched antigenic peptide comprises or consists of an epitope being selected from the group consisting of SEQ ID NOS: 22-23, if said given HLA haplotype is A*2601;
vi. said matched antigenic peptide comprises or consists of an epitope being SEQ ID NO: 24, if said given HLA haplotype is A*3001;
vii. said matched antigenic peptide comprises or consists of an epitope being selected from the group consisting of SEQ ID NOs: 25-26, if said given HLA haplotype is A*6801;
viii. said matched antigenic peptide comprises or consists of an epitope being selected from the group consisting of SEQ ID NOS: 29-37, if said given HLA haplotype is B*0702;
ix. said matched antigenic peptide comprises or consists of an epitope being selected from the group consisting of SEQ ID NO: 38 and SEQ ID NOS: 40-43, if said given HLA haplotype is B*0801;
x. said matched antigenic peptide comprises or consists of an epitope being SEQ ID NO: 44, if said given HLA haplotype is B*1801;
xi. said matched antigenic peptide comprises or consists of an epitope being SEQ ID NO: 45, if said given HLA haplotype is B*3701;
xii. said matched antigenic peptide comprises or consists of an epitope being selected from the group consisting of SEQ ID NOS: 47-48 and SEQ ID NO: 50-52, if said given HLA haplotype is B*4402;
xiii. said matched antigenic peptide comprises or consists of an epitope being selected from the group consisting of SEQ ID NOs: 53-57, if said given HLA haplotype is B*5101;
xiv. said matched antigenic peptide comprises or consists of an epitope being SEQ ID NO: 58 if said given HLA haplotype is C*0202;
xv. said matched antigenic peptide comprises or consists of an epitope being selected from the group consisting of SEQ ID NOs: 59-60 if said given HLA haplotype is C*0304;
xvi. said matched antigenic peptide comprises or consists of an epitope being selected from the group consisting of SEQ ID NOS: 61-66 if said given HLA haplotype is C*0401;
xvii. said matched antigenic peptide comprises or consists of an epitope being selected from the group consisting of SEQ ID NOs: 67-71 if said given HLA haplotype is C*0501;
xviii. said matched antigenic peptide comprises or consists of an epitope being selected from the group consisting of SEQ ID NOs: 72-73 if said given HLA haplotype is C*0701; and/or
xix. said matched antigenic peptide comprises or consists of an epitope being selected from the group consisting of SEQ ID NOs: 74-76 if said given HLA haplotype is C*0702.

In a further embodiment, the kit includes an expansion solution. In a further embodiment, the kit includes instructions for use.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1: T Cell Recognition of LTA and STA Epitopes in MCC Patients

### Aim

Identification of LTA and STA epitopes from MCC patients.

### Material & Methods

Here is described how the LTA and STA epitopes listed in the sequence list were selected based on both an in silico MHC binding prediction and screening for T cell recognition across several MCC patient cohorts.

An extensive library of potential CD8+ T cell epitopes was created, which contained peptides originating from STA and truncated LTA (including their shared region CT) (Genbank: FJ173809.1), and viral capsid protein-1 (VP1) only found on the intact virus (Genbank: FJ173815.1), and therefore served as a control

The in silico peptide prediction of 9- and 10-mer peptides using NetMHCpan 4.0 (Jurtz et al. 2017) resulted in a library of 1490 peptide-MHC complexes with binding capacities to 33 different HLA haplotypes, of which 714 of them presented LTA or STA-derived peptides. All peptides were purchased from Pepscan Presto BV and dissolved to 10 mM in DMSO.

Peripheral blood mononuclear cell (PBMC) or tumor infiltrating lymphocytes (TIL) samples from in total 36 MCC patients (between 1-4 samples per patient) across 3 different cohorts (two cohorts treated with checkpoint inhibitors and one untreated cohort) were screened using DNA barcode-labelled pMHC multimers (Bentzen et al., 2016).

Briefly, biotinylated peptide-MHC (pMHC) complexes were generated for all selected peptides using either UV-induced peptide exchange with MHC monomers folded with UV-sensitive ligands (Hadrup et al. 2009; Rodenko et al. 2006; Toebes et al. 2006) or empty peptide-receptive MHC molecules (Saini et al. 2019). Each pMHC complex was multimerized on a streptavidin-conjugated, PE-labelled dextran backbone carrying a unique DNA barcode, which thereby annotated each pMHC specificity. Hence generating panels of DNA barcode-labelled multimers.

The patient samples were first stained with a mixed pool of HLA-matched multimers for 15 min at 37 ° C., followed by and an antibody mix consisting of CD8-BV480, dump channel antibodies (CD4, CD14, CD19, CD40, CD16) (BD Bioscience), and a viability dye (LIVE/DEAD Fixable Near-IR, Thermo Fischer) for 30 min at 4° C.

Multimer-binding CD8+ T cells were selected and sorted based on their positive PE signal on a FACSAria Fusion (BD Biosciences), pelleted by centrifugation, and cryopreserved at −20° C. The associated DNA barcodes in the pellet were amplified and sequenced to reveal the T cells' peptide specificities. Sequencing data were processed by the software package Barracoda, available online (http://www.cbs.dtu.dk/services/barracoda). DNA barcodes enriched in the sorted

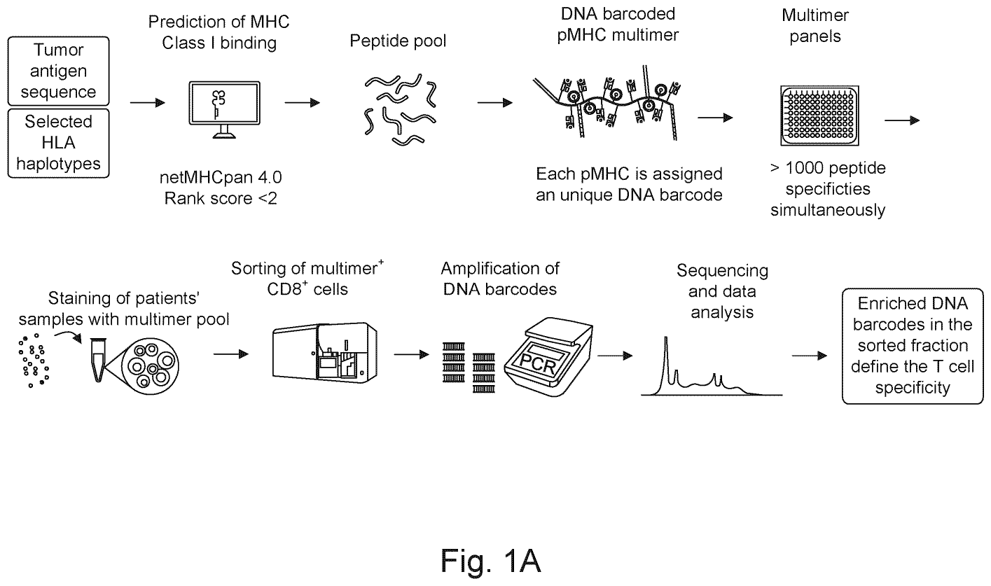

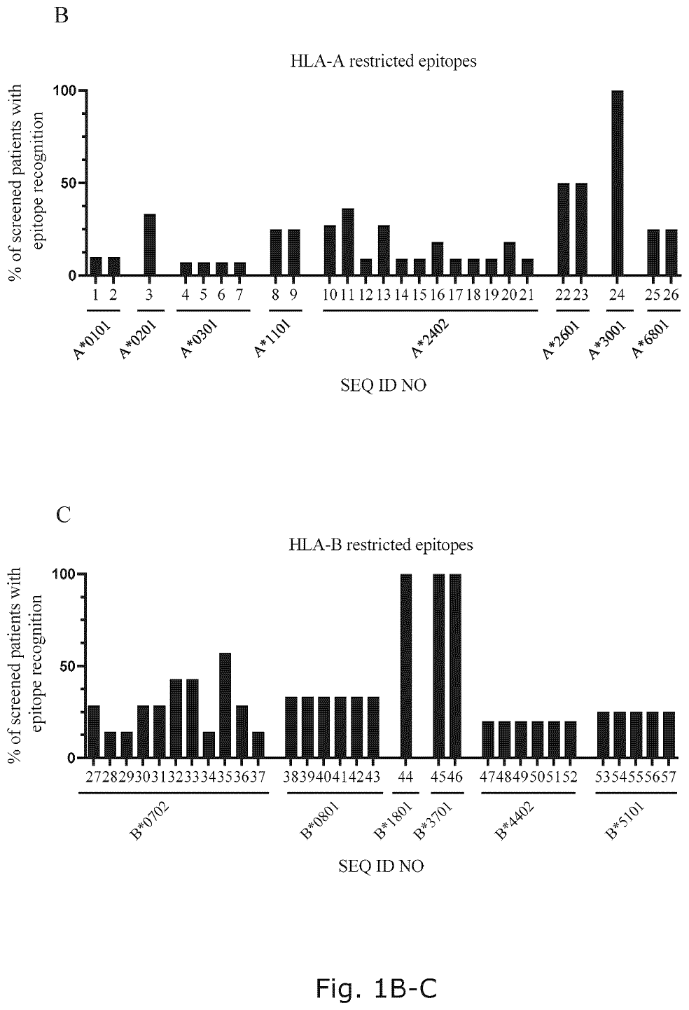

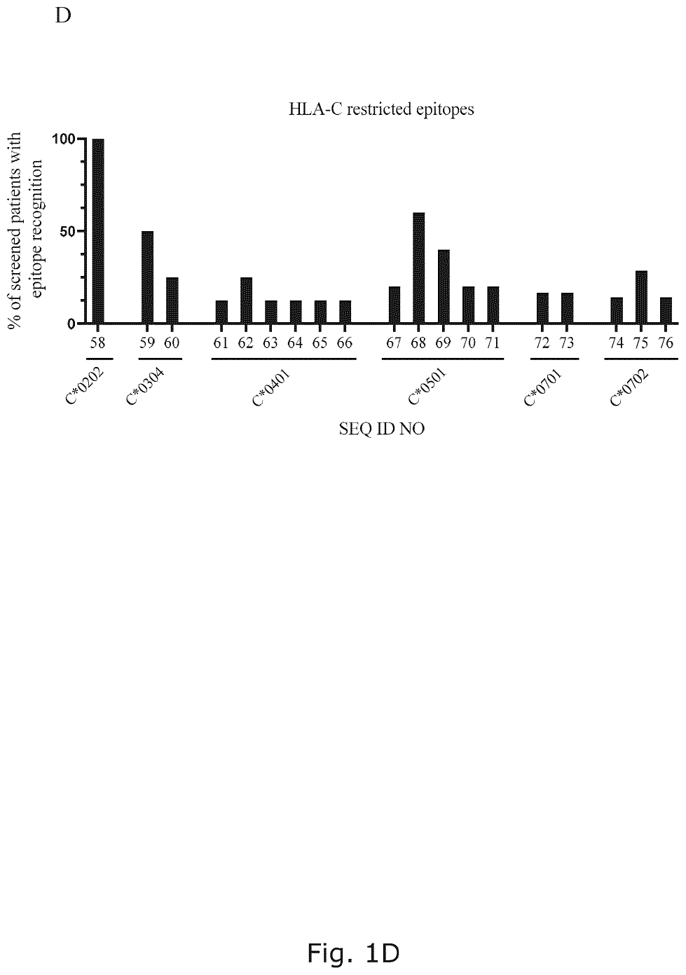

T cell fraction compared to baseline level with an FDR<0.1% were defined as being recognized in the given sample. A schematic overview of the screening process is shown in FIG. 1A.

### Results

The comprehensive HLA haplotype inclusion led to an average HLA coverage of 73% of the patients' HLAs. This large screening effort resulted in a collection of 76 LTA or STA epitopes recognized by T cells across all patients. The epitopes were presented in the context of 20 different MHC haplotypes and were detected in 7.1-100% of MCC patients screened for a given HLA haplotype (FIG. 1B-D, divided into HLA-A, -B, or -C restrictions). The majority of these epitopes were novel ( 67/76) and restricted to new HLA haplotypes not previously investigated ( 10/20).

### Conclusion

Thereby substantially increasing the potential HLA coverage when utilizing these epitopes as targets for antigen-driven T cell expansion strategies.

### Example 2: Epitope Mapping in LTA and STA

### Aim

Mapping of the epitopes from example 1 in the genome of the Merkel Cell polyomavirus.

### Materials & Methods

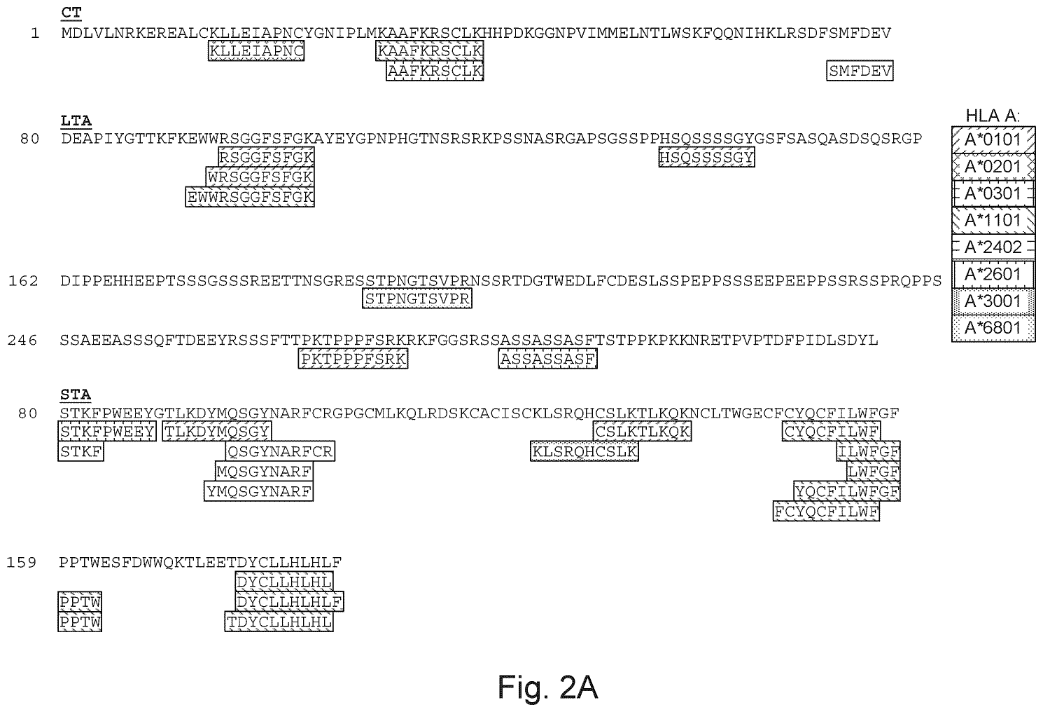

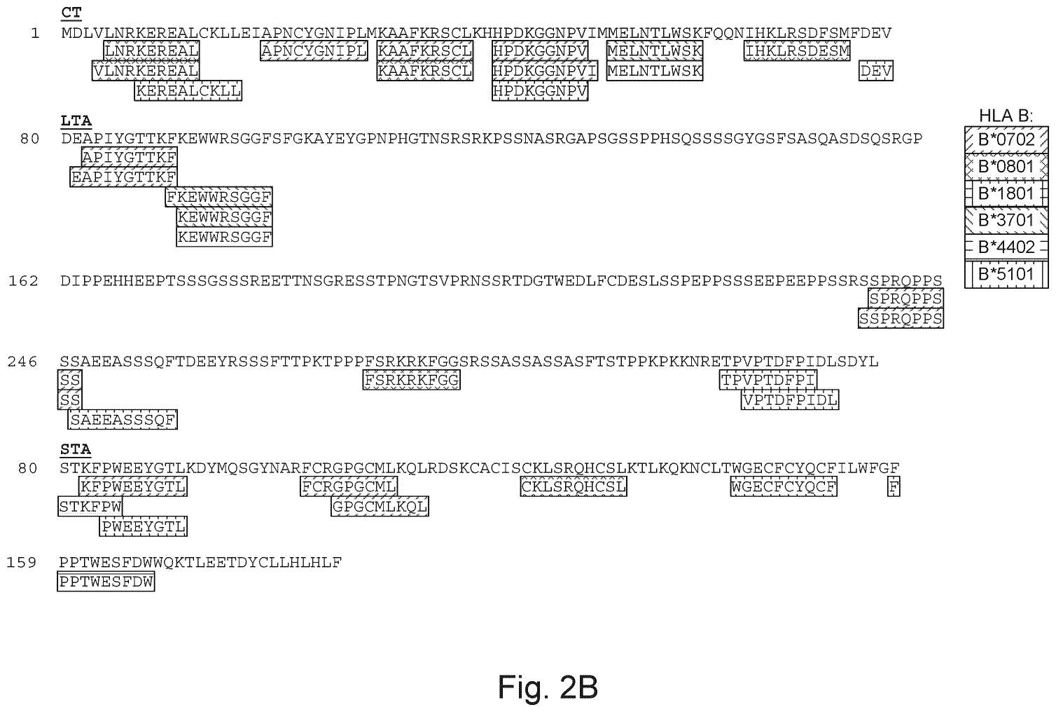

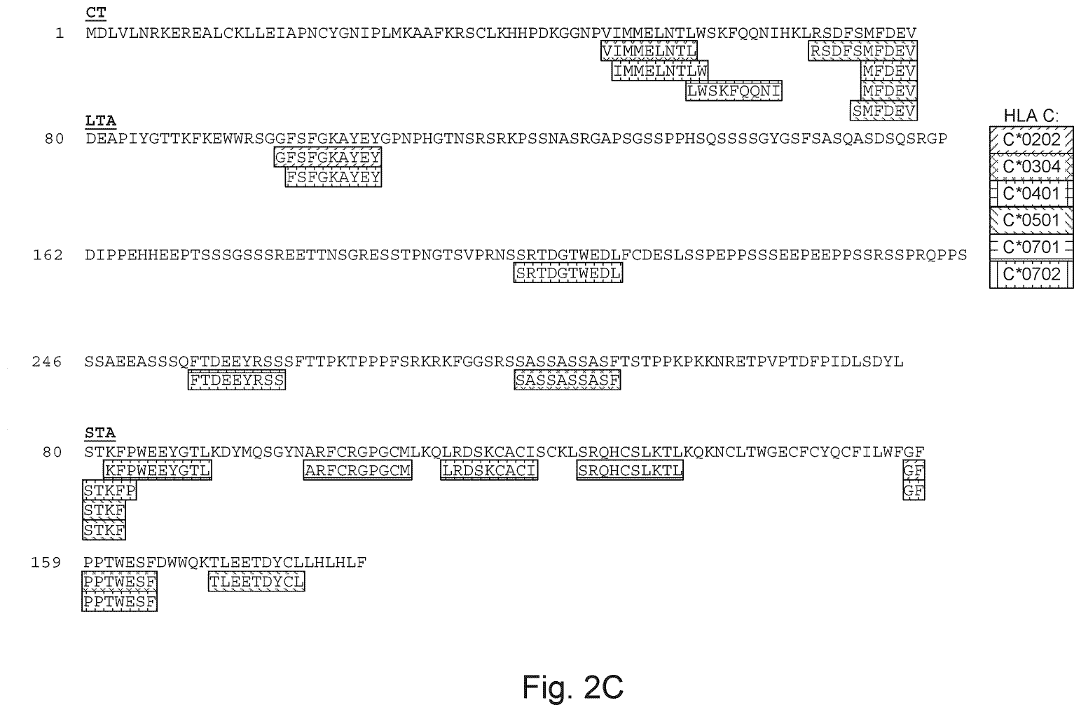

The sequences of the epitopes detected using the DNA barcode-labelled MHC multimer screen (example 1) were mapped along the sequences of truncated LTA (SEQ ID NO: 77), and STA (SEQ ID NO: 78), including their shared CT region (SEQ ID NO: 79) of the genome of the Merkel Cell polyomavirus (FIG. 2A-C, A: HLA-A restricted, B: HLA-B restricted, C: HLA-C restricted). For demonstrating the epitopes, the same sequence is repeated below the sequence in black several times.

### Results

The epitopes are clustering into immunological hotspots where overlapping sequences restricted to the same or even different HLA haplotypes are to be found.

Most strikingly are the clusters along CT, early LTA, and STA, where large overlap can be observed. The peptides found within such clusters are highly likely to be recognized by T cells.

### Conclusion

The epitopes identified in MCC patients cluster along CT, LTA and STA independent of restrictions to either HLA-A, HLA-B or HLA-C.

### Example 3: T Cell Recognition of LTA and STA Epitope in MCC Patients Compared to Healthy Donor

### Aim

To identify the recognition of the LTA and STA epitopes by T cells in MCC patients and healthy donors.

Materials & methods

A cohort of 54 healthy donors was screened identically to the MCC patients as described in example 1, to investigate the T cell recognition of LTA and STA and the control protein VP1.

### Results

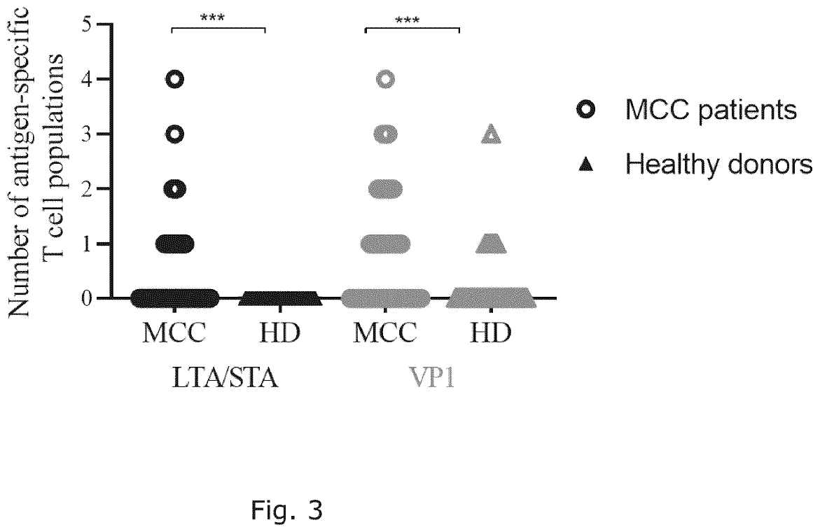

A direct comparison between the two cohorts in terms of number of antigen-specific T cell populations is shown in FIG. 3.

No LTA/STA-specific T cells were detected in the healthy donors (HD), whereas several of these populations were present in the MCC patient cohort (MCC) (FIG. 3, left).

T cells specific for the capside protein VP1 were detected in both cohorts, although significantly more present in the patient cohort (FIG. 3, right).

The VP1 protein is a surface protein presented on the intact virus with up to 88% of the general population showing antibodies against this protein. It is therefore expected that T cells specific for VP1 are present throughout the population. In contrast, the two oncogenes LTA and STA are solely expressed in the tumor and thus only present in cancer patients with MCC.

### Conclusion

These results demonstrated that LTA and STA form unique, cancer-specific T-cell targets that drive tumor recognition in MCC and therefore highly relevant targets for antigen-driven T cell expansion strategies.

### Example 4: Expansion of LTA/STA-Specific T Cell Using Magnetic Cell Sorting and In Vitro Expansion With Anti-CD3/CD28 Expander Beads

### Aim

To demonstrate expansion of CD8+ T cells using APC and epitopes identified in example 1.

### Material & Method

In this example is described how LTA or STA-specific T cells can be expanded from MCC patients' PBMC samples using a magnetic selection of antigen-specific cells.

A limited library of potential CD8+ T cell epitopes was created, due to limitation in the fluorescent-based detection method used. Peptides with binding capacity to one of 9 selected HLA haplotypes were selected based on in silico prediction and further reduced based on previously described T cell recognition. This library therefore consisted of 146 peptide-MHC complexes. All peptides were purchased from Pepscan Presto BV and dissolved to 10 mM in DMSO.

Biotinylated pMHC complexes were generated for all selected LTA/STA-derived peptides using either UV-induced peptide exchange or empty peptide-receptive MHC molecules. All pMHC complexes were multimerized to streptavidin-conjugated PE fluorochrome to create PE-labeled pMHC tetramers.

The tetramers were mixed with PBMCs according to the patients' HLA haplotype for 1 h at 4° C., followed by coupling to anti-PE magnetic beads (Miltenyi Biotec) for 15 min at 4° C. The cells were applied to a magnetic separation column (Miltenyi Biotec) with a 30-mm pre-separation filter (Miltenyi Biotec) placed in a magnetic field for selection of tetramer binding cells.

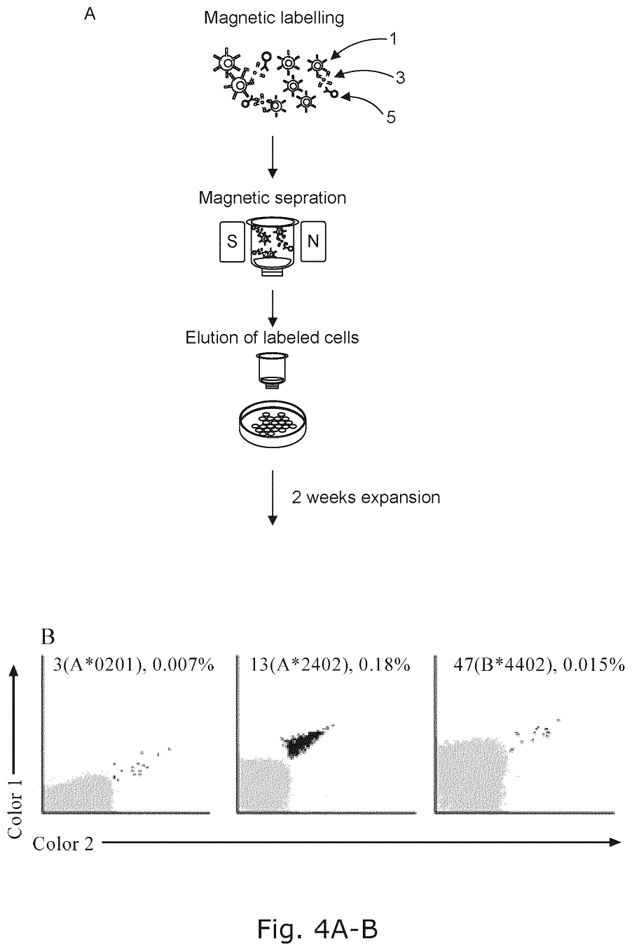

The unbound cell fraction was collected and irradiated at 20 Gy for 15 min to be used as feeder cells. The bound cell fraction was eluted out and cultured 5000 cells/well in a 96-well plate in X-vivo™ 15 supplemented with 5% human serum in the presence of 11×10³ anti-CD3/CD28 Dynabeads, 5×10⁴ feeder cells, 15 ng/ml IL-15, and 100 U/mL IL-2 (FIG. 4A).

The cells were expanded for two weeks with media changed twice a week. The T cells' peptide specificity was revealed using combinatorically encoded fluorescently labeled pMHC tetramers for which all pMHC complexes have been multimerized on two different streptavidin-conjugated fluorochromes (Andersen et al. 2012). Thereby creating unique two-fluorescent color combinations for each pMHC specificity to obtain dual-color positive T cells detectable by flow cytometry.

The cells were stained with an HLA-matched mix of such combinatorically encoded fluorescently labeled pMHC tetramers for 15 min at 37° C., followed by an antibody mix consisting of CD8 and dump channel antibodies (CD4, CD14, CD19, CD40, CD16) and a viability dye (LIVE/DEAD Fixable Near-IR, Thermo Fischer) for 30 min at 4° C. The cells were then analyzed on a LSRFortessa (BD Bioscience).

### Results

Representative dot plots of such expanded LTA/STA-specific T cells are shown in FIG. 4B.

The LTA/STA-specific expanded T cells recognized SEQ ID NO 3, 13, and 47 restricted to three different HLA haplotypes, A*0201, A*2402 and B*4402, respectively. The T cells were of low frequencies, ranging between 0.007-0.2% of total CD8+ T cells even after pMHC based enrichment.

### Conclusion

This experiment demonstrated that it was feasible to expand LTA/STA-specific CD8+ T cells in a peptide-MHC directed manner according to the MCC patients' haplotype.

### Example 5: Expansion of LTA/STA-Specific T Cell Using Artificial Antigen Presenting Cell Scaffolds

### Aim

To demonstrate expansion of LTA or STA-specific T cells from PBMCs from an MCC patient using aAPC scaffolds.

### Material & Methods

Prior to expansion, the patient samples were screened with DNA barcode-labelled multimers as described in example 1 to elucidate the epitope-specificity of the LTA/STA-specific T cells. Based on the screen the A*2402 restricted peptides; CYQCFILWF (SEQ ID NO 10, STA), DYCLLHLHL (SEQ ID NO 12, STA), and EWWRSGGFSF (SEQ ID NO 13, LTA) were selected and used to create aAPC scaffolds.

Briefly, biotinylated pMHC complexes were generated for the selected peptides (see example 4) using empty peptide-receptive MHC molecules i.e. A*2402. The aAPC scaffolds were assembled by mixing unlabeled streptavidin-conjugated dextran with the pMHC complex, biotinylated IL-2, and biotinylated IL-21 in a stichometry of 1:24:6:6 (dextran: pMHC: IL-2: IL-21). The aAPC scaffold was allowed to assemble at 4ºC for half an hour and was thereafter purified and separated from unbound molecules through a cut-off filter Vivaspin 6 centrifugal concentrator, MWCO 100 kDa. The assembled aAPC scaffold was kept at −20° C. until addition to the cell culture.

On day 0, the cell culture was established from the patient's PBMCs and initiated with 1.7×10⁶ cells in 1 ml X-vivo™ 15 media supplemented with 5% human serum in a 48-well flat bottom culture plate. The aAPC scaffold was added to the culture and incubated for 1 hour at 37° C. and 5% CO₂. Hereafter the cell culture was transferred to a 24-well G-rex plate, topped up to 5 ml X-vivo™ 15 media supplemented with 5% human serum, and left at 37° C. and 5% CO₂.

On day 3, 6, and 9, half of the media was changed and the cells were stimulated with the aAPC scaffolds. On day 14, the cells were harvested and the peptide specificity was revealed by staining with single-color fluorescently labeled pMHC tetramers for 15 min at 37° C. and an antibody mix consisting of CD8, CD3, (BD Bioscience) and a viability dye (LIVE/DEAD Fixable Near-IR, Thermo Fischer) for 30 min at 4° C. The cells were then analyzed on a LSRFortessa (BD Bioscience).

### Results

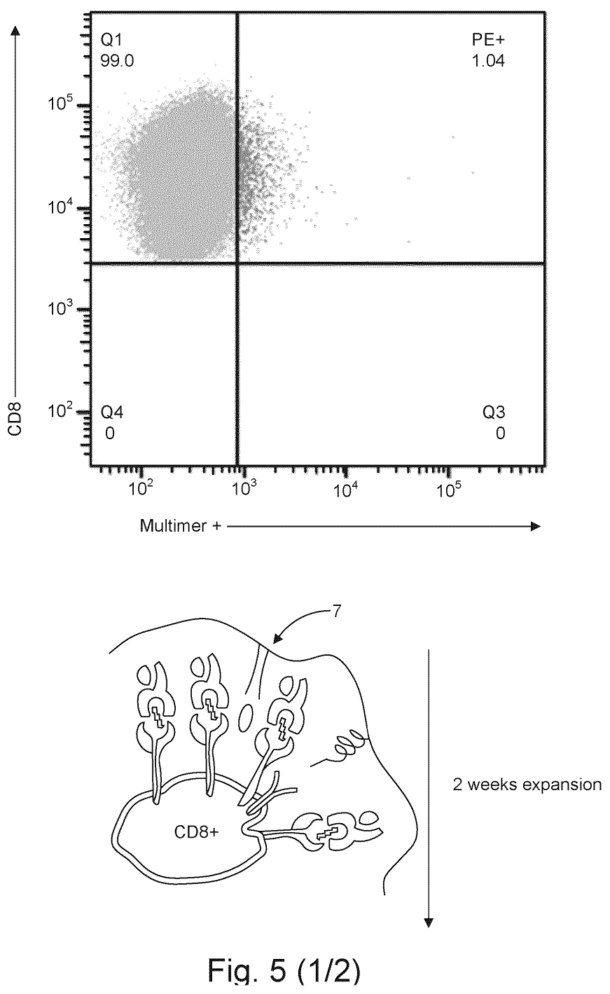

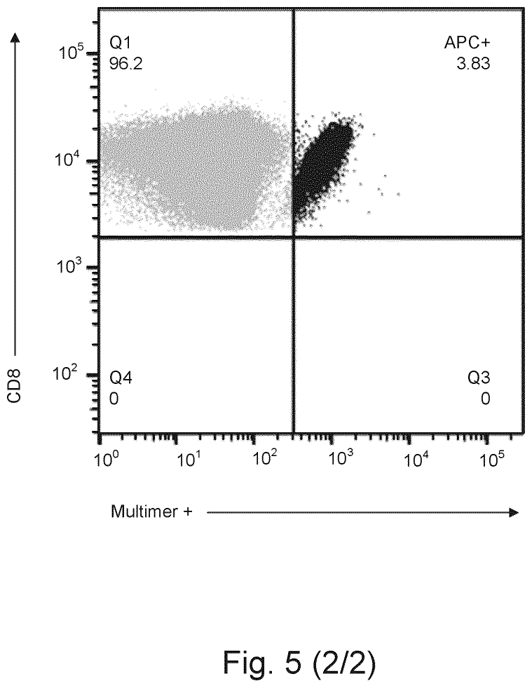

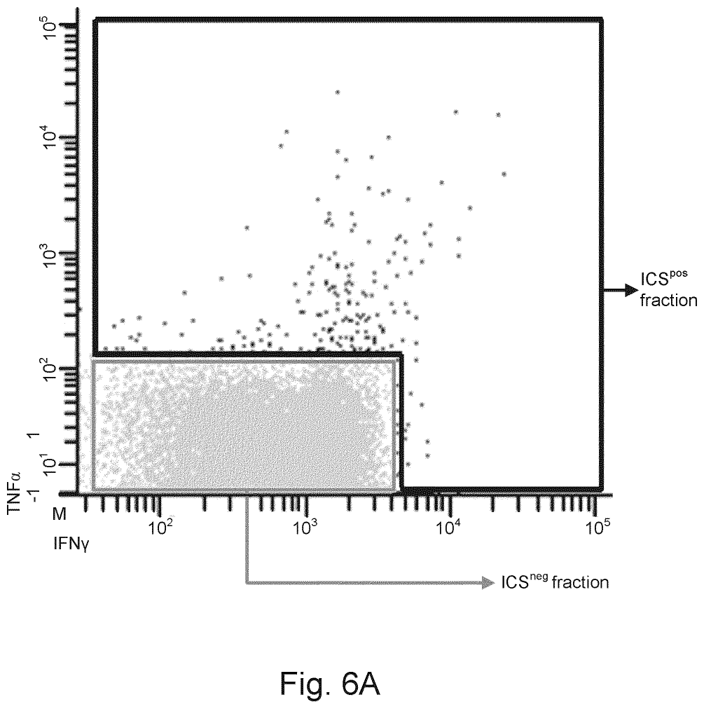

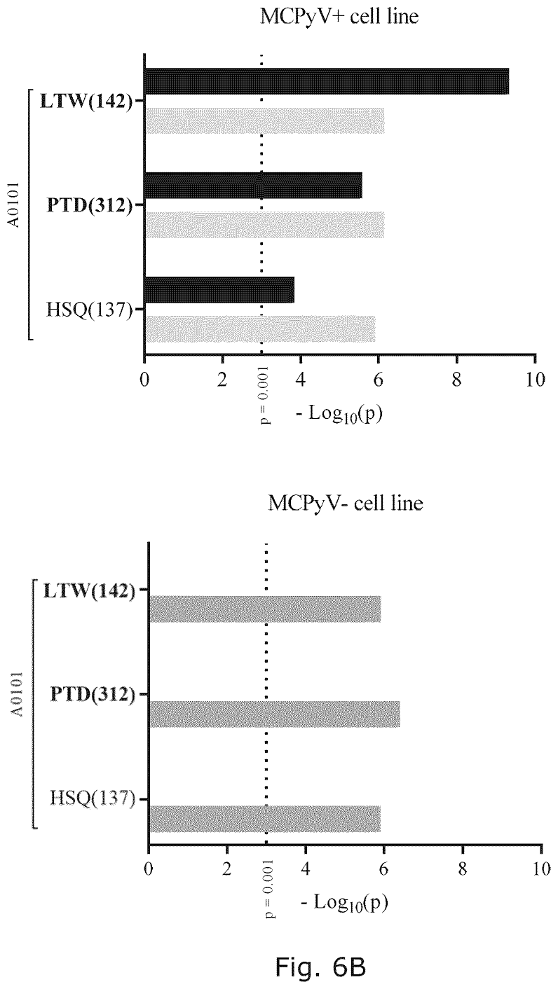

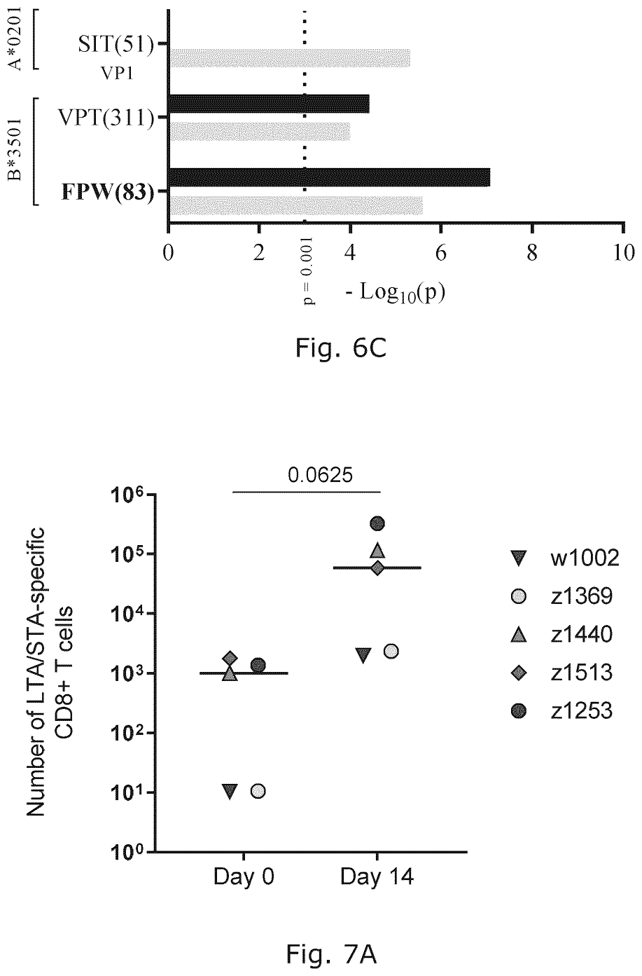

Dot plots from both the DNA barcode-labelled multimer screen and the expanded T cells are shown in FIG. 5. The top dot plot (FIG. 5 (½)) shows the 1.04% of CD8+ T cells that were positive for the DNA-barcoded multimers and sorted during the DNA barcode-labelled multimer screen.

From these, the peptide-specificity was revealed by sequencing of the associated DNA barcodes and showed that less than 0.045% of total CD8+ was LTA/STA-specific cells.

Following the expansion with aAPC scaffolds, the T cells recognizing the epitope EWWRSGGFSF (SEQ ID NO 13, LTA) was now accounting for 3.83% of total CD8+ T cells (dot plot in FIG. 5 ( 2/2))). Thereby demonstrating that this expansion strategy is capable of expanding LTA/STA-specific T cells from very low frequencies.

Compared to the expansion with magnetic selection of antigen-specific cells described in the previous example, the frequency of specific cells was substantially higher with this expansion strategy.

### Conclusion

This experiment demonstrated that it is possible to expand LTA/STA CD8+ T cells from very low frequencies.

### Example 6: Expansion of LTA/STA-Specific T Cell Using moDC

### Aim

To demonstrate expansion of LTA or STA-specific T cells from PBMCs from an MCC patient using monocyte-derived (mo)DC.

### Materials & Methods

7 days prior to expansion MoDCs are generated from the MCC patient's PBMC. The PBMCs are plated out in a 6-well plate at a density of 2-3*10\hat{ }6/cm² in Monocyte Attachment Medium (PromoCell). The cells are incubated for 1 hour at 37° C. and 5% CO₂ to allow the monocytes to attach. All other cells are then washed off by vigorously swirling the plate to loosen non-adherent cells and aspirate the supernatant. The adherent cells are washed three times with warm Monocyte Attachment Medium (PromoCell) by swirling the plates and aspirating the supernatant. Dendritic Cell Generation Medium supplemented with 1× Component A of the Cytokine Pack (PromoCell) is added to the immature moDC and left for 3 days at 37° C. and 5% CO₂.

On day 3, the medium is changed by aspirating the supernatant into a collection tube to prevent any cell loss and immediately, pipetting fresh PromoCell DC Generation Medium supplemented with 1× Component A of the Cytokine Pack (PromoCell) to the cells in the plate. The collection tube is centrifuged for 10 min at 180×g, and the supernatant removed and the cells are transferred back to the plate and left for 3 days at 37° C. and 5% CO₂.

On day 6, the moDC maturation process is completed by supplementing the cells with 1× of Component B of the Cytokine Pack (PromoCell) and leaving them for 24 hours at 37° C. and 5% CO₂.

On day 7, the mature moDCs are harvested and prepared for T cells expansion. The moDCs are plated out in a 96-well round-bottom plate at a density of 1*10\hat{ }6 cells/well in X-vivo™ 15 supplemented with 5% human serum. The moDCs are pulsed with 5 μM selected LTA/STA peptides and incubated for 4 hours at room temperature. The remaining moDCs are cryopreserved. During the incubation, the PBMCs from the patient are prepared and plated out in a 24-well G-rex plate in 5 ml X-vivo™ 15 supplemented with 5% human serum, 100 IU/ml IL-2, and 50 ng/ml IL-21. The pulsed moDCs are washed once and transferred to the G-rex plate to obtain a ratio of 1:10 (moDC:PBMC), and the cells are left for 7 days at 37° C. and 5% CO₂.

On day 7 of the T cell expansion, the cells are restimulated with a second round of moDCs. The moDCs are plated out in a 96-well round-bottom plate at a density of 1*10\hat{ }6 cells/well in X-vivo™ 15 supplemented with 5% human serum. The moDCs are pulsed with 5 μM selected LTA/STA peptides and incubated for 4 hours at room temperature. The pulsed moDCs are washed once and transferred to the G-rex plate to obtain a ratio of 1:10 (moDC:PBMC), and the cells were left for 7 days at 37° C. and 5% CO₂.

On day 14 of the T cell expansion, the cells are harvested and the peptide specificity is revealed by staining with combinatorically encoded fluorescently labeled pMHC tetramers for 15 min at 37° C. and an antibody mix consisting of CD8, CD3, (BD Bioscience) and a viability dye (LIVE/DEAD Fixable Near-IR, Thermo Fischer) for 30 min at 4° C. The cells are then analyzed on a LSRFortessa (BD Bioscience).

### Results & Conclusion

The results will demonstrate that it is possible to generate patient derived moDCs and use them to expand LTA/STA CD8+ T cells.

### Example 7: MCC Tumor Cells Recognition Using Expanded LTA/STA-Specific T Cells

### Aim

To describe the functional capacity of LTA/STA-specific T cells in response to MCC tumor cell recognition through an assay combining profiling of T-cell specificity and functionality.

### Material & Methods

In this case, the cytokines TNFα and IFNγ were used as markers for cytotoxic activated T cells upon target recognition. Such tumor cell recognition will depend on both the functional profile of the T cells and the correct epitope processing and presentation in the context of MHC class I on the tumor cell surface.

PBMC samples from two MCC patients were expanded using the magnetic selection of antigen-specific cells combined with CD3/CD28 expander beads as described in example 4.

HLA-matched Merkel Cell Polyomavirus (MCPyV)-positive or -negative tumor cell lines (TCLs) were pre-stimulated with 250 U/mL IFNγ for 24 hours at 37° C. and 5% CO₂ prior to co-culture in order to increase MHC class I presentation. 1*10\hat{ }6 expanded PBMCs were mixed with 1*10\hat{ }5 tumor cells in a 10:1 ratio (PBMC: TCL) in X-vivo media supplemented with 5% human serum in a 96-well plate and incubated for 4 hours at 37° C. and 5% CO₂.

DNA barcode-labeled pMHC multimers were generated for all relevant HLA-matched peptides selected in example 4.

Following the co-culture, the cells were first stained with DNA barcode-labeled pMHC multimers for 15 min at 37° C. and then relevant cell surface antibodies (CD3, CD8, and viability dye) for 30 min at 4° C. Afterward the cells were permeabilized with a permeabilization buffer and stained for intracellular cytokines (TNFα and IFNγ) for 30 min at 4° C.

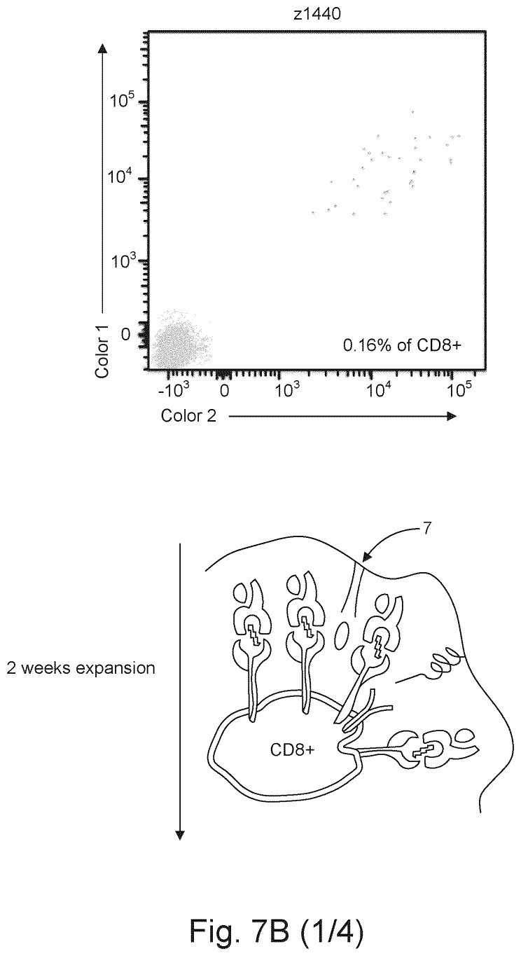

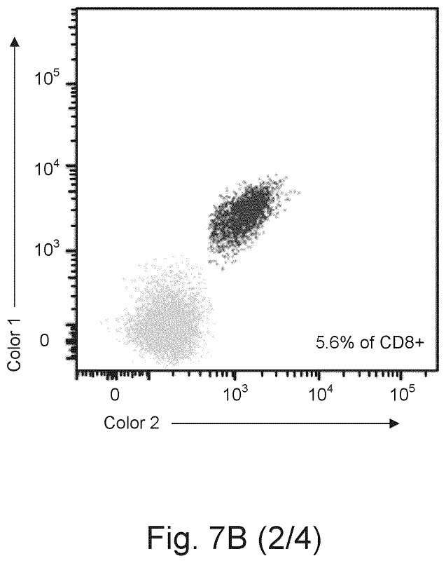

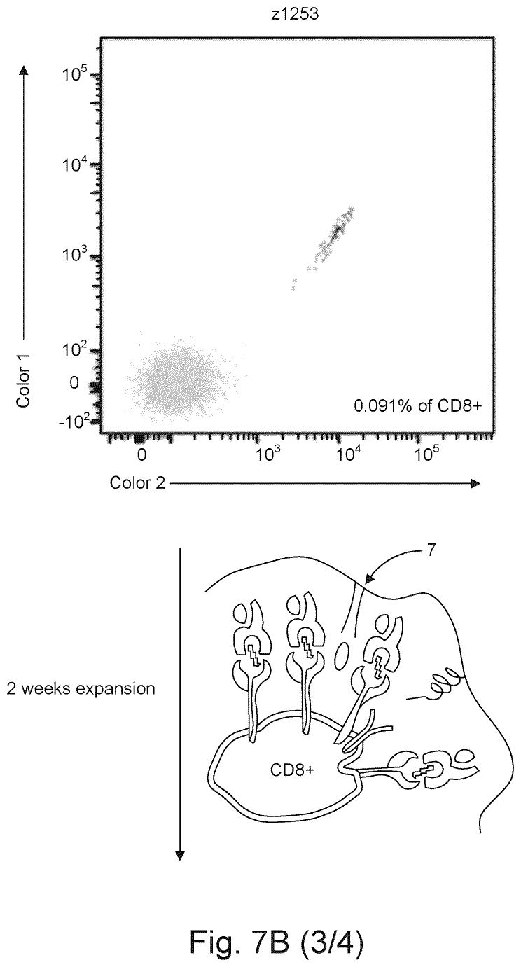

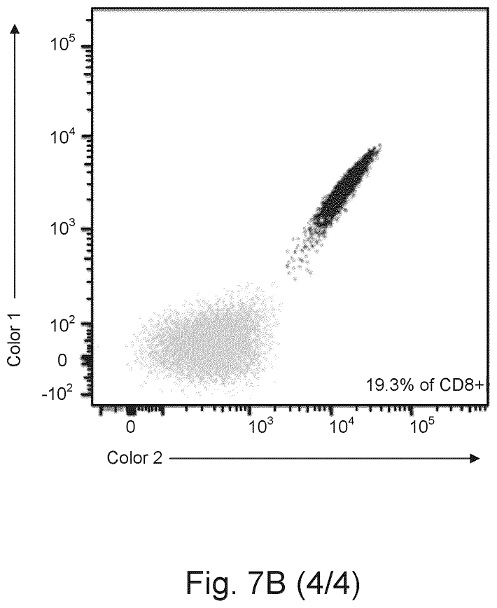

The cells were then sorted on a FACSAria Fusion according to their cytokine secretion profile (IFNγ and/or TNFα secretion (ICS^{pos}) or no cytokine secretion (ICS^{neg})) as described in Bentzen et al., 2016 (FIG. 7A). For both the ICS^{pos} and ICS^{neg} fraction, the pMHC specificity was revealed by sequencing of the associated DNA barcode.

The co-attached DNA barcodes present in both sorted pools were amplified and sequenced in order to reveal the epitope specificities of the functionally activated cells using the software package Barracoda, available online (http://www.cbs.dtu.dk/services/barracoda).

Presented as bar plots displaying −Log₁₀(p) of significantly enriched DNA barcodes, hence pMHC specificities recognized by T cells in either the ICS^{pos} fraction (black bars) and the ICS^{neg} fraction (grey bars) (FIG. 7B-C).

### Results

This experiment demonstrated that LTA/STA-specific T cells were functionally activated upon tumor cell stimulation.

In PBMCs from patient #1 three T cell populations were activated upon MCPyV+TCL stimulation. These T cells recognized HLA-A*0101-restricted LTA/STA-derived epitopes (FIG. 7B, top).

When the same cells were stimulated with a MCPyV−TCL, which is an MCC tumor without expression of the viral oncogenes originating from virus-negative MCC, no T cells are functionally activated. Still, the same three LTA/STA-specific T cell populations are detected in the ICS^{neg} fraction (FIG. 7B, bottom). Demonstrating a lack of T-cell responsiveness when LTA and STA are not available, and hence that these antigens are highly relevant tumor targets in T cell mediated anti-tumor cytotoxicity.

In PBMCs from patient #2, two T cell populations are activated upon MCPyV+TCL stimulation, recognizing two HLA-B*3501-restricted LTA/STA-derived epitopes (FIG. 7C). Again, demonstrating that these antigens are the target for tumor cell recognition.

In addition, a VP1-derived epitope was recognized in the ICS^{neg} fraction but did not result in functional activation of the T cells. In line with the knowledge that no VP1 expression is expected in the tumor, and hence this protein is not a mediator for tumor cell recognition.

Unfortunately, no MCPyV−TCL was available matching the HLA haplotypes of this patient.

For both patients, the activated T-cell populations were also detected in the ICS^{neg} fraction, which indicates either partial functionality of the T cells (potentially caused by the expansion strategy used) or incomplete stimulation based on the TCLs antigen presentation.

### Conclusion

These experiments demonstrated that the antigens tested for HLA-A*0101 and HLA-B*3501 are targets for tumor cell recognition. Thus, the HLA haplotype restricted epitopes can be used for stimulation and expansion of T cell composition directed towards MCC.

### Example 8: Expansion of LTA/STA-Specific T Cell Using Artificial Antigen Presenting Cell Scaffolds

### Aim

To demonstrate expansion of LTA or STA-specific T cells from PBMCs from MCC patients using person-tailored aAPC scaffolds based on the patients' HLA haplotypes.

### Material & Methods

The HLA haplotypes of 5 MCC patients (between 1-4 HLA haplotypes per patient) were used to create person-tailored aAPC scaffolds. The HLA haplotypes of patient z1440 were A*0201, A*2402 and B*0702, which were combined with the peptides SEQ ID NO: 3, SEQ ID NOS: 10-21 and SEQ ID NOS: 27-37, respectively. For patient z1369, the HLA haplotypes A*0101, A*0201 and B*0801 were combined with the peptides SEQ ID NOs: 1-2, SEQ ID NOS: 3 and SEQ ID NOs: 38-43, respectively. For patient z1513, the HLA haplotypes A*2402 and B*0702 were combined with the peptides SEQ ID NOs: 10-21, and SEQ ID NOs: 27-37, respectively. For patient w1002, the HLA haplotypes A*0101, A*0301 and B*0702 were combined with the peptides SEQ ID NOS: 1-2, SEQ ID NOS: 4-7 and SEQ ID NOs: 27-37, respectively. For patient z1253, a single HLA haplotype A*0301 was combined with the peptides SEQ ID NOs: 4-7.

Briefly, biotinylated pMHC complexes were generated for the selected peptides using either UV-induced peptide exchange or empty peptide-receptive MHC molecules. The aAPC scaffolds were assembled by mixing unlabeled streptavidin-conjugated dextran with the pMHC complex, biotinylated IL-2, and biotinylated IL-21 in a stichometry of 1:24:6:6 (dextran: pMHC: IL-2: IL-21). The aAPC scaffold was allowed to assemble at 4° C. for half an hour and was thereafter purified and separated from unbound molecules through a cut-off filter Vivaspin 6 centrifugal concentrator, MWCO 100 kDa. The assembled aAPC scaffold was kept at −20° C. until addition to the cell culture.

On day 0, the cell culture was established from the patient's PBMCs and initiated with 5-10×10⁶ cells in 1 ml X-vivo™ 15 media supplemented with 5% human serum in a 24-well G-rex plate. The aAPC scaffold was added to the culture and incubated for 1 hour at 37° C. and 5% CO₂. Hereafter the cell culture was topped up to 5 ml X-vivo™ 15 media supplemented with 5% human serum and left at 37° C. and 5% CO₂.

On day 3, 6, and 9, half of the media was changed and the cells were stimulated with the aAPC scaffolds. On day 14, the cells were harvested and the peptide specificities of the expanded and un-expanded cells were revealed by staining with combinatorially encoded fluorescently labeled pMHC tetramers (see example 4) for 15 min at 37° C. and an antibody mix consisting of CD8, CD3, (BD Bioscience) and a viability dye (LIVE/DEAD Fixable Near-IR, Thermo Fischer) for 30 min at 4° C. The cells were then analyzed on a LSRFortessa (BD Bioscience).

### Results

The LTA/STA-specific expansions of the 5 patients' PBMCs are summarized in FIG. 7. The graph (FIG. 7A) shows the total number of LTA/STA-specific CD8+ T cells detectable/estimated at day 0 (un-expanded cells) and day 14 (expanded cells) for each of the 5 patients (w1002, z1369, z1440, z1513 and z1253, respectively). For two of the patients, no LTA/STA-specific cells were detected at day 0. Instead, an estimation was made where the number of cells at the 0.001% detection limit of the combinatorically encoded fluorescently labeled pMHC tetramers were used. The average number of LTA/STA-specific CD8+ T cells was 834 cells on day 0 and 100814 on day 14. This corresponded to an average fold change in LTA/STA-specific cells of 159.

Representative dot plots from two single expansions are shown in FIG. 7B. In patient z1440 the A*2402 restricted epitope CYQCFILWF (SEQ ID NO 10, STA) was expanded from 0.16% to 5.6% of CD8+ T cells (FIG. 7B (¼- 2/4)). Resulting in a 137-fold change in the number of epitope-specific T cells.

Similar, in patient z1253 the A*0301 restricted epitope RSGGFSFGK (SEQ ID NO 6, LTA) was expanded from 0.091% to 19.3% of CD8+ T cells (FIG. 7B (¾- 4/4)). This was a 452-fold change in the number of epitope-specific T cells.

### Conclusion

This experiment demonstrated that it is possible to expand LTA/STA CD8+ T cells with person-tailored aAPC scaffolds without prior detection of peptide specificity. The expansion strategy enabled expansion of LTA/STA-specific T cells from even undetectable levels and with high fold changes in the number of specific cells.

### Example 9: Phenotype of LTA/STA-Specific T Cells Expanded With Artificial Antigen Presenting Cell (aAPC) Scaffolds

### Aim

To evaluate the phenotype of LTA or STA-specific T cells after expansion with person-tailored aAPC scaffolds.

### Material & Methods

Expanded and un-expanded PBMCs from the 5 MCC patients described in example 8 were used to evaluate possible phenotypic changes after the expansion with person-tailored aAPC scaffolds.

The cells were first stained with combinatorial encoded fluorescently labeled pMHC tetramers for 15 min at 37° C. where all peptide specificities had been given the same dual-color combination. Followed by an antibody mix consisting of CD8, CD3, CD45RA, CD28, CD39 (BD Bioscience), PD-1, CD27, CCR7 (BioLegend), and a viability dye (LIVE/DEAD Fixable Near-IR, Thermo Fischer) for 30 min at 4° C. The cells were then fixated with a fixation/permeabilization buffer (eBioscience™ Foxp3/Transcription Factor Staining Buffer Set, Invitrogen) for 30 min at 4° C. before they were permeabilized with permeabilization buffer (eBioscience™ Foxp3/Transcription Factor Staining Buffer Set, Invitrogen) and stained with an intracellular/intranuclear antibody mix consisting of TCF1, Ki67 (BD Bioscience), TOX (Miltenyi Biotec) and GZMb (BioLegend) for 60 min at room temperature. The cells were then analyzed on a LSRFortessa (BD Bioscience).

### Results

The phenotypes of the 5 patients' PBMCs are shown in FIG. 8. The pooled tetramer-specific cells were first analyzed based on their expression of CCR7 and CD45RA to define them as either effector memory (EM), central memory (CM), terminally differentiated effector memory (TEMRA), or naïve T cells. The un-expanded cells consisted of a mix of all cell types with EM being the most dominant. Whereas the aAPC scaffold expanded cells were solely EM T cells, which are functionally activated cells (FIG. 8A).

The pooled tetramer-specific cells could then be further analyzed for the other 8 phenotype markers (FIG. 8B) to compare un-expanded with expanded cells. The expanded cells showed a more favorable phenotype with higher activation levels given by CD27, CD28, and CD39 expression, increased proliferation by Ki67, and increased cytotoxic activity with GZMb expression. Whereas the un-expanded cells showed higher levels of the exhaustion marker TOX.

### Conclusion

These results demonstrated that the T cells expanded with aAPC scaffolds showed an activated and proliferative phenotype with high cytotoxic activity compared to un-expanded cells.

### Example 10: MCC Tumor Cell Killing Using aAPC Scaffold Expanded Cells

### Aim

To define the capacity for tumor cell killing among LTA or STA-specific T cells after expansion with person-tailored aAPC scaffolds or un-expanded PBMCs.

### Material & Methods

Expanded and un-expanded PBMCs from one of the MCC patients described in example 8 were used as effector cells in the Flow cytotox killing assay where a co-culture between true target cells, non-target cells and effector cells can be used to evaluate the specific lysis of the true target cells.

Prior to the co-culture, the HLA-matched MCPyV-positive TCL, WAGA (true target), was pre-stimulated with 250 U/mL IFNγ for 24 hours at 37° C. and 5% CO₂ in order to increase MHC class I presentation.

The irrelevant HLA-mismatching cell line T2 was used as non-target cells.

The true target cells were labeled with the dye Carboxyfluorescein succinimidyl ester (CFSE, Fisher Scientific) and the non-target cells were labeled with Far Red (FR, Fisher Scientific) for 5 min at room temperature. The labeling processes were terminated by addition of Fetal Bovine Serum (FCS, Fisher Scientific).

Co-culture wells were set up with 50000 true target and non-target cells and mixed with effector cells at different effector:true target ratios in RPMI media (Fisher Scientific) supplemented with 10% FCS (Fisher Scientific) and 10 IU/mL IL-2.

Target-only wells were set up by only adding true target and non-target cells in RPMI media (Fisher Scientific) supplemented with 10% FCS (Fisher Scientific) and IU/mL IL-2. All cultures were left at 37° C. and 5% CO₂ for 48 hours.

Hereafter, the cells could directly be analyzed on Attune NXT Flow Cytometer (Thermo Fischer) to measure the amount of the two cell labeling dyes. The ratio between the dyes was then compared between co-culture wells and target-only wells in order to calculate specific lysis of true target cells.

### Results

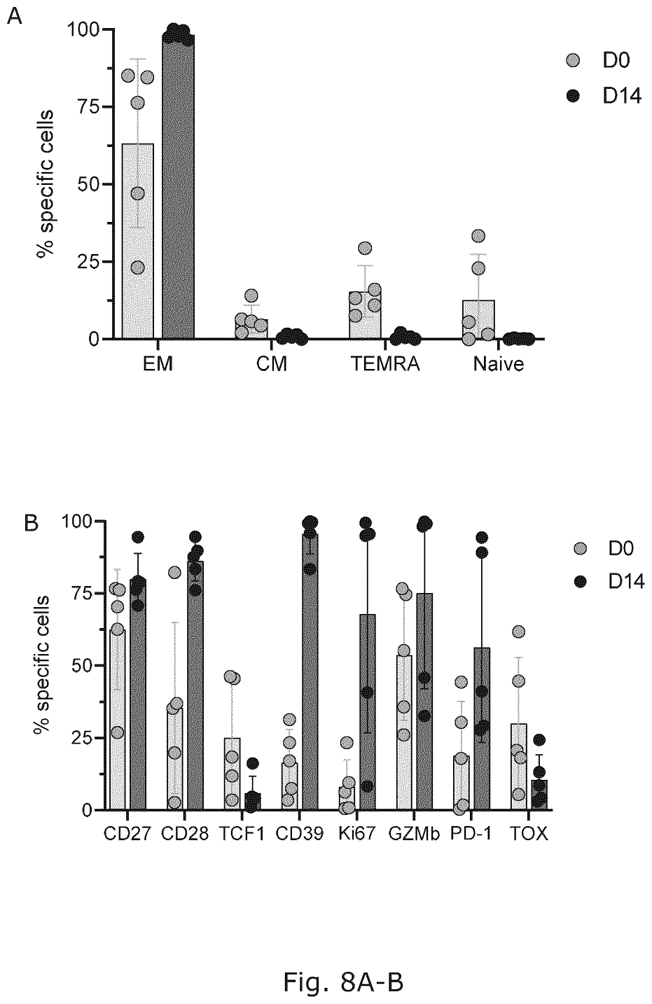

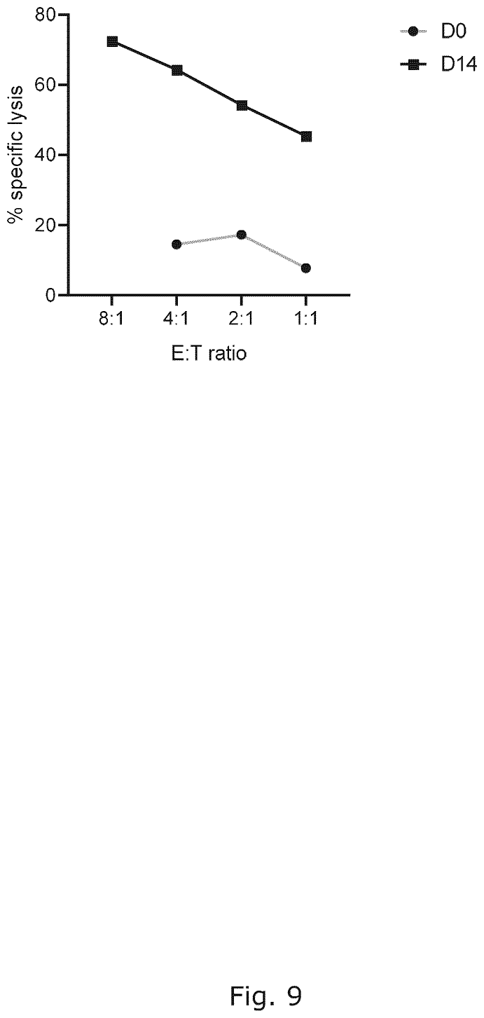

The graph in FIG. 9 shows in grey the specific lysis of true target (WAGA) after co-culture with the un-expanded PBMCs. It ranged from 9-17% depending on the effector:target ratio (E:T ratio). In black is the specific lysis detected after co-culture with the T cells expanded using aAPC scaffolds, where it was feasible to do effector:target ratios up to 8:1. The specific lysis of WAGA ranged from 46-73%.

Comparing the specific lysis between the expanded and un-expanded cells at E:T ratios 1:1-4:1, showed on average a 4.5-fold higher specific lysis using the expanded cells. These data are in agreement with the phenotype data presented in example 9, which showed a favorable phenotype of the LTA/STA-specific T cells expanded with aAPC scaffolds with increased cytotoxic activity compared to un-expanded cells.

### Conclusion

This experiment demonstrated that the person-tailored expansion with aAPC scaffolds presenting LTA/STA epitopes increased the killing capacity of the T cells against the allogeneic tumor cell line (WAGA) compared to un-expanded PBMCs.

TABLE 1
Match between HLA haplotype and epitope as
well as the

| Matched | MCV | Sequence of | SEQ |
|---|---|---|---|
| HLA | Protein | epitope | ID NO |
| A*0101 | LTA | HSQSSSSGY | 1 |
| A*0101 | STA | TLKDYMQSGY | 2 |
| A*0201 | CT | KLLEIAPNC | 3 |
| A*0301 | STA | CSLKTLKQK | 4 |
| A*0301 | LTA | PKTPPPFSRK | 5 |
| A*0301 | LTA | RSGGFSFGK | 6 |
| A*0301 | LTA | WRSGGFSFGK | 7 |
| A*1101 | CT | KAAFKRSCLK | 8 |
| A*1101 | STA | QSGYNARFCR | 9 |
| A*2402 | STA | CYQCFILWF | 10 |
| A*2402 | STA | DYCLLHLHL | 11 |
| A*2402 | STA | DYCLLHLHLF | 12 |
| A*2402 | LTA | EWWRSGGFSF | 13 |
| A*2402 | STA | ILWFGFPPTW | 14 |
| A*2402 | STA | LWFGFPPTW | 15 |
| A*2402 | STA | MQSGYNARF | 16 |
| A*2402 | STA* | SMFDEVSTKF | 17 |
| A*2402 | STA | TDYCLLHLHL | 18 |
| A*2402 | STA | YMQSGYNARF | 19 |
| A*2402 | STA | YQCFILWFGF | 20 |
| A*2402 | STA | FCYQCFILWF | 21 |
| A*2601 | LTA | ASSASSASF | 22 |
| A*2601 | STA | STKFPWEEY | 23 |
| A*3001 | STA | KLSRQHCSLK | 24 |
| A*6801 | LTA | STPNGTSVPR | 25 |
| A*6801 | CT | AAFKRSCLK | 26 |
| B*0702 | LTA | APIYGTTKF | 27 |
| B*0702 | CT | APNCYGNIPL | 28 |
| B*0702 | LTA | EAPIYGTTKF | 29 |
| B*0702 | STA | FCRGPGCML | 30 |
| B*0702 | STA | GPGCMLKQL | 31 |
| B*0702 | CT | HPDKGGNPV | 32 |
| B*0702 | CT | HPDKGGNPVI | 33 |
| B*0702 | STA | KFPWEEYGTL | 34 |
| B*0702 | CT | KAAFKRSCL | 35 |
| B*0702 | LTA | SPRQPPSSS | 36 |
| B*0702 | LTA | SSPRQPPSSS | 37 |
| B*0801 | LTA | FSRKRKFGG | 38 |
| B*0801 | CT | KAAFKRSCL | 39 |
| B*0801 | CT | LNRKEREAL | 40 |
| B*0801 | CT | VLNRKEREAL | 41 |
| B*0801 | CT | IHKLRSDFSM | 42 |
| B*0801 | STA | CKLSRQHCSL | 43 |
| B*1801 | CT | MELNTLWSK | 44 |
| B*3701 | LTA | FKEWWRSGGF | 45 |
| B*3701 | LTA | KEWWRSGGF | 46 |
| B*4402 | STA* | DEVSTKFPW | 47 |
| B*4402 | CT | KEREALCKLL | 48 |
| B*4402 | LTA | KEWWRSGGF | 49 |
| B*4402 | CT | MELNTLWSK | 50 |
| B*4402 | LTA | SAEEASSSQF | 51 |
| B*4402 | STA | WGECFCYQCF | 52 |
| B*5101 | STA | FPPTWESFDW | 53 |
| B*5101 | STA | FPWEEYGTL | 54 |
| B*5101 | CT | HPDKGGNPV | 55 |
| B*5101 | LTA | TPVPTDFPI | 56 |
| B*5101 | LTA | VPTDFPIDL | 57 |
| C*0202 | LTA | GFSFGKAYEY | 58 |
| C*0304 | LTA | SASSASSASF | 59 |
| C*0304 | CT | VIMMELNTL | 60 |
| C*0401 | STA | GFPPTWESF | 61 |
| C*0401 | CT | IMMELNTLW | 62 |
| C*0401 | STA | KFPWEEYGTL | 63 |
| C*0401 | STA* | MFDEVSTKFP | 64 |
| C*0401 | STA | LRDSKCACI | 65 |
| C*0401 | CT | LWSKFQQNI | 66 |
| C*0501 | LTA | FTDEEYRSS | 67 |
| C*0501 | STA* | MFDEVSTKF | 68 |
| C*0501 | CT | RSDFSMFDEV | 69 |
| C*0501 | STA* | SMFDEVSTKF | 70 |
| C*0501 | STA | TLEETDYCL | 71 |
| C*0701 | STA | ARFCRGPGCM | 72 |
| C*0701 | STA | SRQHCSLKTL | 73 |
| C*0702 | LTA | FSFGKAYEY | 74 |
| C*0702 | STA | GFPPTWESF | 75 |
| C*0702 | LTA | SRTDGTWEDL | 76 |

*Sequence overlaps the CT region and non-CT region of the STA

### SEQUENCE LISTING

| | |
|---|---|
| SEQ ID NO: 1-76 | see table 1 |
| SEQ ID NO: 77 | truncated LTA i.e. LTA without CT |
| | region (FIG. 2A-C) |
| SEQ ID NO: 78 | truncated STA i.e. STA without CT |
| | region (FIG. 2A-C) |
| SEQ ID NO: 79 | CT region; part of both LTA and |
| | STA (FIG. 2A-C) |

### REFERENCES

Andersen R S, Kvistborg P, Frøsig T M, Pedersen N W, Lyngaa R, Bakker A H, et al. Parallel detection of antigen-specific T cell responses by combinatorial encoding of MHC multimers. Nat. Protoc. 2012; 7(5): 891-902
Bentzen A K, Marquard A M, Lyngaa R, Saini S K, Ramskov S, Donia M, et al. Large-scale detection of antigen-specific T cells using peptide-MHC-I multimers labeled with DNA barcodes. Nat. Biotechnol. 2016; 34(10):1037-45
Jurtz V, Paul S, Andreatta M, Marcatili P, Peters B, Nielsen M. NetMHCpan-4.0: Improved Peptide-MHC Class I Interaction Predictions Integrating Eluted Ligand and Peptide Binding Affinity Data. J Immunol. 2017 Nov. 1; 199(9):3360-8
Hadrup S R, Toebes M, Rodenko B, Bakker A H, Egan D, Huib O, et al. High-throughput T-cell epitope discovery through MHC peptide exchange. Methods Mol Biol. 2009; 524:383-405
Ichikawa J, Yoshida T, Isser A, Laino A S, Vassallo M, Woods D, et al. Rapid Expansion of Highly Functional Antigen-Specific T Cells from Patients with Melanoma by Nanoscale Artificial Antigen-Presenting Cells. Clin Cancer Res. 2020; 26(13): 3384-3396.
Rodenko B, Toebes M, Hadrup S R, van Esch W J E, Molenaar A M, Schumacher T N M, et al. Generation of peptide-MHC class I complexes through UV-mediated ligand exchange. Nat. Protoc. 2006; 1(3):1120-32
Toebes M, Coccoris M, Bins A, Rodenko B, Gomez R, Nieuwkoop N J, et al. Design and use of conditional MHC class I ligands. Nat. Med. 2006; 12:246-51
Saini, S K, Tamhane T, Anjanappa R, Saikia A, Ramskov S, Donia M et al. Empty peptide-receptive MHC class I molecules for efficient detection of antigen-specific T cells. Sci. Immunol. 2019; 4(37): DOI: 10.1126/sciimmunol.aau9039.
Silva D A, Yu S, Ulge U Y, Spangler J B, Jude K M, Labão-Almeida C, et al. De novo design of potent and selective mimics of IL-2 and IL-15. Nature 2019; 565, 186-191.

1. A method for producing a person-tailored T cell composition by in vitro stimulation and expansion of T cells comprising:
i. providing at least one identified HLA haplotype from a subject;
ii. preparing at least one Antigen Presenting Cell (APC) comprising at least one HLA haplotype corresponding to said at least one identified HLA haplotype; and at least one antigenic peptide matched to said at least one HLA haplotype; wherein said at least one antigenic peptide comprises an epitope from Merkel cell polyomavirus, said epitope originating from large T antigen (LTA), small T antigen (STA) or the shared region (CT) of LTA and STA;
iii. providing a sample comprising T cells,
iv. contacting said sample with an expansion solution comprising at least one APC as prepared in step ii,
iv. stimulating and expanding T cells with specificity for said at least one antigenic peptide comprised on at least one APC in culture, and, optionally harvesting the T cells from the culture, to obtain a person-tailored T cell composition.

2-15. (canceled)

16. The method according to claim 1, wherein:
i. said at least one antigenic peptide comprises an epitope selected from the group consisting of SEQ ID NOs: 1 and 2, if said HLA haplotype is A*0101;
ii. said at least one antigenic peptide comprises an epitope being SEQ ID NO: 3, if said HLA haplotype is A*0201;
iii. said at least one antigenic peptide comprises an epitope selected from the group consisting of SEQ ID NOs: 4, 5, 6, and 7, if said HLA haplotype is A*0301;
iv. said at least one antigenic peptide comprises an epitope selected from the group consisting of SEQ ID NOs: 8 and 9, if said HLA haplotype is A*1101;
v. said at least one antigenic peptide comprises an epitope selected from the group consisting of SEQ ID NOs: 10, 11, 12, 13, 14, 15, 16. 17, 18, 19, 20, and 21, if said HLA haplotype is A*2402;
vi. said at least one antigenic peptide comprises an epitope selected from the group consisting of SEQ ID NOs: 22 and 23, if said HLA haplotype is A*2601;
vii. said at least one antigenic peptide comprises an epitope being SEQ ID NO: 24, if said HLA haplotype is A*3001; and/or
viii. said at least one antigenic peptide comprises an epitope selected from the group consisting of SEQ ID NOs: 25 and 26, if said HLA haplotype is A*6801.

17. The method according to claim 1, wherein
i. said at least one antigenic peptide comprises an epitope selected from the group consisting of SEQ ID NOs: 27, 28, 29, 30, 31, 32, 33, 34, 35, 36, and 37, if said HLA haplotype is B*0702;
ii. said at least one antigenic peptide comprises an epitope selected from the group consisting of SEQ ID NOs: 38. 39, 40, 41, 42, and 43, if said HLA haplotype is B*0801;
iii. said at least one antigenic peptide comprises epitope being SEQ ID NO: 44, if said HLA haplotype is B*1801;
iv. said at least one antigenic peptide comprises an epitope selected from the group consisting of SEQ ID NOs: 45 and 46, if said HLA haplotype is B*3701;
v. said at least one antigenic peptide comprises an epitope selected from the group consisting of SEQ ID NOs: 47, 48, 49, 50, 51, and 52, if said HLA haplotype is B*4402; and/or
vi. said at least one antigenic peptide comprises an epitope selected from the group consisting of SEQ ID NOs: 53, 54, 55, 56, and 57, if said HLA haplotype is B*5101.

18. The method according to claim 1, wherein
i. said at least one antigenic peptide comprises an epitope being SEQ ID NO: 58 if said HLA haplotype is C*0202;
ii. said at least one antigenic peptide comprises an epitope selected from the group consisting of SEQ ID NOs: 59 and 60 if said HLA haplotype is C*0304;
iii. said at least one antigenic peptide comprises an epitope selected from the group consisting of SEQ ID NOs: 61, 62, 63, 64, 65, and 66 if said HLA haplotype is C*0401;
iv. said at least one antigenic peptide comprises an epitope selected from the group consisting of SEQ ID NOs: 67, 68, 69, 70, and 71 if said HLA haplotype is C*0501;
v. said at least one antigenic peptide comprises an epitope selected from the group consisting of SEQ ID NOs: 72 and 73 if said HLA haplotype is C*0701; and/or
vi. said at least one antigenic peptide comprises an epitope selected from the group consisting of SEQ ID NOs: 74, 75, and 76 if said HLA haplotype is C*0702.

19. The method according to claim 1, wherein said at least one identified HLA haplotype is identified by providing a biological sample from said subject and identifying at least one HLA haplotype from said biological sample.

20. The method according to claim 19, wherein said biological sample is identical to said sample comprising T cells.

21. The method according to claim 1, wherein said APC is an artificial APC (aAPC), an autologous APC or an allogenic APC.

22. The method according to claim 21, wherein said aAPC is selected from the group consisting of an aAPC scaffold, aAPC beads and a cellular aAPC.

23. The method according to claim 1, wherein the method further comprises separating or sorting the T cells prior to expanding them.

24. The method according to claim 1, wherein the solution comprises a number of different APCs resembling the number of identified HLA haplotypes.

25. A person-tailored T cell composition obtained by a method as described in claim 1.

26. A method of inhibiting or treating Merkel Cell Carcinoma in a subject comprising administering the person-tailored T cell composition produced by the method of claim 1 to said subject.

27. A kit for expansion of T cells, the kit comprising:
i) at least one Antigen Presenting Cell (APC) having a given HLA haplotype;
ii) at least one antigenic peptide, wherein said antigenic peptide comprises an epitope from Merkel cell polyomavirus, said epitope originating from large T antigen (LTA), small T antigen (STA) or the shared region (CT) of LTA and STA; and
wherein said at least one APC and said at least one antigenic peptide are configured to be combined by combining said given HLA haplotype with a matched antigenic peptide.

28. The kit according to claim 27, wherein
i. said matched antigenic peptide comprises or consists of an epitope being SEQ ID NO: 2, if said given HLA haplotype is A*0101;
ii. said matched antigenic peptide comprises an epitope selected from the group consisting of SEQ ID NOs: 4, 5, and SEQ ID NO: 7, if said given HLA haplotype is A*0301;
iii. said matched antigenic peptide comprises an epitope selected from the group consisting of SEQ ID NOs: 8 and 9, if said given HLA haplotype is A*1101;
iv. said matched antigenic peptide comprises an epitope selected from the group consisting of SEQ ID NOs: 10, 11, 12, 14, 15, 16, 17, 18, 19, 20, and 21, if said given HLA haplotype is A*2402;
v. said matched antigenic peptide comprises an epitope selected from the group consisting of SEQ ID NOs: 22 and 23, if said given HLA haplotype is A*2601;
vi. said matched antigenic peptide comprises an epitope being SEQ ID NO: 24, if said given HLA haplotype is A*3001;
vii. said matched antigenic peptide comprises an epitope selected from the group consisting of SEQ ID NOs: 25 and 26, if said given HLA haplotype is A*6801;
viii. said matched antigenic peptide comprises an epitope selected from the group consisting of SEQ ID NOs: 29, 30, 31, 32, 33, 34, 35, 36, and 37, if said given HLA haplotype is B*0702;
ix. said matched antigenic peptide comprises an epitope selected from the group consisting of SEQ ID NO: 38, 40, 41, 42, and 43, if said given HLA haplotype is B*0801;
x. said matched antigenic peptide comprises an epitope being SEQ ID NO: 44, if said given HLA haplotype is B*1801;
xi. said matched antigenic peptide comprises an epitope being SEQ ID NO: 45, if said given HLA haplotype is B*3701;
xii. said matched antigenic peptide comprises an epitope selected from the group consisting of SEQ ID NOs: 47, 48, 50, 51, and 52, if said given HLA haplotype is B*4402;
xiii. said matched antigenic peptide comprises an epitope selected from the group consisting of SEQ ID NOs: 53, 54, 55, 56, and 57, if said given HLA haplotype is B*5101;
xiv. said matched antigenic peptide comprises an epitope being SEQ ID NO: 58 if said given HLA haplotype is C*0202;
xv. said matched antigenic peptide comprises an epitope selected from the group consisting of SEQ ID NOs: 59 and 60 if said given HLA haplotype is C*0304;
xvi. said matched antigenic peptide comprises an epitope selected from the group consisting of SEQ ID NOs: 61, 62, 63, 64, 65, and 66, if said given HLA haplotype is C*0401;
xvii. said matched antigenic peptide comprises an epitope selected from the group consisting of SEQ ID NOs: 67, 68, 69, 70, and 71 if said given HLA haplotype is C*0501;
xviii. said matched antigenic peptide comprises an epitope selected from the group consisting of SEQ ID NOs: 72 and 73 if said given HLA haplotype is C*0701; and/or
xix. said matched antigenic peptide comprises an epitope selected from the group consisting of SEQ ID NOs: 74, 75, and 76 if said given HLA haplotype is C*0702.


SEQUENCE LISTING (79 sequences)

SEQ ID NO: 1 - protein, 9 aa, Merkel cell polyomavirus
HSQSSSSGY

SEQ ID NO: 2 - protein, 10 aa, Merkel cell polyomavirus
TLKDYMQSGY

SEQ ID NO: 3 - protein, 9 aa, Merkel cell polyomavirus
KLLEIAPNC

SEQ ID NO: 4 - protein, 9 aa, Merkel cell polyomavirus
CSLKTLKQK

SEQ ID NO: 5 - protein, 10 aa, Merkel cell polyomavirus
PKTPPPFSRK

SEQ ID NO: 6 - protein, 9 aa, Merkel cell polyomavirus
RSGGFSFGK

SEQ ID NO: 7 - protein, 10 aa, Merkel cell polyomavirus
WRSGGFSFGK

SEQ ID NO: 8 - protein, 10 aa, Merkel cell polyomavirus
KAAFKRSCLK

SEQ ID NO: 9 - protein, 10 aa, Merkel cell polyomavirus
QSGYNARFCR

SEQ ID NO: 10 - protein, 9 aa, Merkel cell polyomavirus
CYQCFILWF

SEQ ID NO: 11 - protein, 9 aa, Merkel cell polyomavirus
DYCLLHLHL

SEQ ID NO: 12 - protein, 10 aa, Merkel cell polyomavirus
DYCLLHLHLF

SEQ ID NO: 13 - protein, 10 aa, Merkel cell polyomavirus
EWWRSGGFSF

SEQ ID NO: 14 - protein, 10 aa, Merkel cell polyomavirus
ILWFGFPPTW

SEQ ID NO: 15 - protein, 9 aa, Merkel cell polyomavirus
LWFGFPPTW

SEQ ID NO: 16 - protein, 9 aa, Merkel cell polyomavirus
MQSGYNARF

SEQ ID NO: 17 - protein, 10 aa, Merkel cell polyomavirus
SMFDEVSTKF

SEQ ID NO: 18 - protein, 10 aa, Merkel cell polyomavirus
TDYCLLHLHL

SEQ ID NO: 19 - protein, 10 aa, Merkel cell polyomavirus
YMQSGYNARF

SEQ ID NO: 20 - protein, 10 aa, Merkel cell polyomavirus
YQCFILWFGF

SEQ ID NO: 21 - protein, 10 aa, Merkel cell polyomavirus
FCYQCFILWF

SEQ ID NO: 22 - protein, 9 aa, Merkel cell polyomavirus
ASSASSASF

SEQ ID NO: 23 - protein, 9 aa, Merkel cell polyomavirus
STKFPWEEY

SEQ ID NO: 24 - protein, 10 aa, Merkel cell polyomavirus
KLSRQHCSLK

SEQ ID NO: 25 - protein, 10 aa, Merkel cell polyomavirus
STPNGTSVPR

SEQ ID NO: 26 - protein, 9 aa, Merkel cell polyomavirus
AAFKRSCLK

SEQ ID NO: 27 - protein, 9 aa, Merkel cell polyomavirus
APIYGTTKF

SEQ ID NO: 28 - protein, 10 aa, Merkel cell polyomavirus
APNCYGNIPL

SEQ ID NO: 29 - protein, 10 aa, Merkel cell polyomavirus
EAPIYGTTKF

SEQ ID NO: 30 - protein, 9 aa, Merkel cell polyomavirus
FCRGPGCML

SEQ ID NO: 31 - protein, 9 aa, Merkel cell polyomavirus
GPGCMLKQL

SEQ ID NO: 32 - protein, 9 aa, Merkel cell polyomavirus
HPDKGGNPV

SEQ ID NO: 33 - protein, 10 aa, Merkel cell polyomavirus
HPDKGGNPVI

SEQ ID NO: 34 - protein, 10 aa, Merkel cell polyomavirus
KFPWEEYGTL

SEQ ID NO: 35 - protein, 9 aa, Merkel cell polyomavirus
KAAFKRSCL

SEQ ID NO: 36 - protein, 9 aa, Merkel cell polyomavirus
SPRQPPSSS

SEQ ID NO: 37 - protein, 10 aa, Merkel cell polyomavirus
SSPRQPPSSS

SEQ ID NO: 38 - protein, 9 aa, Merkel cell polyomavirus
FSRKRKFGG

SEQ ID NO: 39 - protein, 9 aa, Merkel cell polyomavirus
KAAFKRSCL

SEQ ID NO: 40 - protein, 9 aa, Merkel cell polyomavirus
LNRKEREAL

SEQ ID NO: 41 - protein, 10 aa, Merkel cell polyomavirus
VLNRKEREAL

SEQ ID NO: 42 - protein, 10 aa, Merkel cell polyomavirus
IHKLRSDFSM

SEQ ID NO: 43 - protein, 10 aa, Merkel cell polyomavirus
CKLSRQHCSL

SEQ ID NO: 44 - protein, 9 aa, Merkel cell polyomavirus
MELNTLWSK

SEQ ID NO: 45 - protein, 10 aa, Merkel cell polyomavirus
FKEWWRSGGF

SEQ ID NO: 46 - protein, 9 aa, Merkel cell polyomavirus
KEWWRSGGF

SEQ ID NO: 47 - protein, 9 aa, Merkel cell polyomavirus
DEVSTKFPW

SEQ ID NO: 48 - protein, 10 aa, Merkel cell polyomavirus
KEREALCKLL

SEQ ID NO: 49 - protein, 9 aa, Merkel cell polyomavirus
KEWWRSGGF

SEQ ID NO: 50 - protein, 9 aa, Merkel cell polyomavirus
MELNTLWSK

SEQ ID NO: 51 - protein, 10 aa, Merkel cell polyomavirus
SAEEASSSQF

SEQ ID NO: 52 - protein, 10 aa, Merkel cell polyomavirus
WGECFCYQCF

SEQ ID NO: 53 - protein, 10 aa, Merkel cell polyomavirus
FPPTWESFDW

SEQ ID NO: 54 - protein, 9 aa, Merkel cell polyomavirus
FPWEEYGTL

SEQ ID NO: 55 - protein, 9 aa, Merkel cell polyomavirus
HPDKGGNPV

SEQ ID NO: 56 - protein, 9 aa, Merkel cell polyomavirus
TPVPTDFPI

SEQ ID NO: 57 - protein, 9 aa, Merkel cell polyomavirus
VPTDFPIDL

SEQ ID NO: 58 - protein, 10 aa, Merkel cell polyomavirus
GFSFGKAYEY

SEQ ID NO: 59 - protein, 10 aa, Merkel cell polyomavirus
SASSASSASF

SEQ ID NO: 60 - protein, 9 aa, Merkel cell polyomavirus
VIMMELNTL

SEQ ID NO: 61 - protein, 9 aa, Merkel cell polyomavirus
GFPPTWESF

SEQ ID NO: 62 - protein, 9 aa, Merkel cell polyomavirus
IMMELNTLW

SEQ ID NO: 63 - protein, 10 aa, Merkel cell polyomavirus
KFPWEEYGTL

SEQ ID NO: 64 - protein, 10 aa, Merkel cell polyomavirus
MFDEVSTKFP

SEQ ID NO: 65 - protein, 9 aa, Merkel cell polyomavirus
LRDSKCACI

SEQ ID NO: 66 - protein, 9 aa, Merkel cell polyomavirus
LWSKFQQNI

SEQ ID NO: 67 - protein, 9 aa, Merkel cell polyomavirus
FTDEEYRSS

SEQ ID NO: 68 - protein, 9 aa, Merkel cell polyomavirus
MFDEVSTKF

SEQ ID NO: 69 - protein, 10 aa, Merkel cell polyomavirus
RSDFSMFDEV

SEQ ID NO: 70 - protein, 10 aa, Merkel cell polyomavirus
SMFDEVSTKF

SEQ ID NO: 71 - protein, 9 aa, Merkel cell polyomavirus
TLEETDYCL

SEQ ID NO: 72 - protein, 10 aa, Merkel cell polyomavirus
ARFCRGPGCM

SEQ ID NO: 73 - protein, 10 aa, Merkel cell polyomavirus
SRQHCSLKTL

SEQ ID NO: 74 - protein, 9 aa, Merkel cell polyomavirus
FSFGKAYEY

SEQ ID NO: 75 - protein, 9 aa, Merkel cell polyomavirus
GFPPTWESF

SEQ ID NO: 76 - protein, 10 aa, Merkel cell polyomavirus
SRTDGTWEDL

SEQ ID NO: 77 - protein, 244 aa, Merkel cell polyomavirus
DEAPIYGTTKFKEWWRSGGFSFGKAYEYGPNPHGTNSRSRKPSSNASRGAPSGSSPPHSQSSSSGYGSFSASQASDSQSRGPDIPPEHHEEPTSSSGSSSREETTNSGRESSTPNGTSVPRNSSRTDGTWEDLFCDESLSSPEPPSSSEEPEEPPSSRSSPRQPPSSSAEEASSSQFTDEEYRSSSFTTPKTPPPFSRKRKFGGSRSSASSASSASFTSTPPKPKKNRETPVPTDFPIDLSDYL

SEQ ID NO: 78 - protein, 107 aa, Merkel cell polyomavirus
STKFPWEEYGTLKDYMQSGYNARFCRGPGCMLKQLRDSKCACISCKLSRQHCSLKTLKQKNCLTWGECFCYQCFILWFGFPPTWESFDWWQKTLEETDYCLLHLHLF

SEQ ID NO: 79 - protein, 79 aa, Merkel cell polyomavirus
MDLVLNRKEREALCKLLEIAPNCYGNIPLMKAAFKRSCLKHHPDKGGNPVIMMELNTLWSKFQQNIHKLRSDFSMFDEV